(19) United States
(12) Patent Application Publication
(10) Pub. No.: US 2023/0235077 A1
(43) Pub. Date: Jul. 27, 2023

(54) MATERIALS AND METHODS OF TREATING CANCER

(71) Applicants: THE GENERAL HOSPITAL CORPORATION, Boston, MA (US); MONOPTEROS THERAPEUTICS, INC., Chestnut Hill, MA (US)

(72) Inventors: Thorsten R. MEMPEL, Chestnut Hill, MA (US); Mauro DI PILATO, Wakefield, MA (US); Ulrich H. VON ANDRIAN, Chestnut Hill, MA (US); Peter KELLER, Chestnut Hill, MA (US)

(73) Assignees: THE GENERAL HOSPITAL CORPORATION, Boston, MA (US); MONOPTEROS THERAPEUTICS, INC., Chestnut Hill, MA (US)

(21) Appl. No.: 18/011,605

(22) PCT Filed: Jun. 24, 2021
(86) PCT No.: PCT/US2021/038880

§ 371 Date: Dec. 20, 2022

Related U.S. Application Data
(60) Provisional application No. 63/043,163, filed on Jun. 24, 2020.

(51) Int. Cl. C07K 16/28; A61K 31/415; A61K 31/4709; A61P 35/00

(52) U.S. Cl. CPC C07K 16/2896; A61K 31/415; A61K 31/4709; A61P 35/00

(57) ABSTRACT

The present disclosure provides a method of treating cancer in a subject in need thereof comprising administering a checkpoint inhibitor to the subject, and administering a MALT-1 inhibitor according to an intermittent dosage regimen.

### BACKGROUND

Solid tumors are infiltrated by effector T cells (Teff) with the potential to control or reject them, as well as by regulatory T cells (Treg) that restrict the function of Teff and thereby promote tumor growth¹. The anti-tumor activity of Teff can be therapeutically unleashed and is now being exploited for the treatment of some select forms of human cancer. However, weak tumor-associated inflammatory responses and the immune-suppressive function of Treg remain major hurdles to broader effectiveness of tumor immunotherapy².

### SUMMARY

In one aspect, described herein is a method of treating cancer in a subject in need thereof comprising (a) administering a checkpoint inhibitor to the subject; (b) administering a first dose of a MALT-1 inhibitor to the subject for up to 7 days; (c) withholding administering the MALT-1 inhibitor for at least 2 days after the administration of (b); and (d) administering a second dose of the MALT-1 inhibitor to the subject after the withholding step.

In some embodiments, the checkpoint inhibitor is an anti-TIM3 antibody, an anti-TIGIT antibody, an anti-VISTA antibody, an anti-LAG3 antibody, an anti-NKG2A antibody, an anti-PD1 antibody, an anti-PD-L1 antibody or an anti-PD-L2 antibody. In some embodiments, the checkpoint inhibitor is an anti-PD1 antibody. Exemplary anti-PD1 antibodies include, but are not limited to, Pembrolizumab (Keytruda), Nivolumab, AUNP-12 and Pidilizumab.

In some embodiments, the checkpoint inhibitor is an anti-PDL1 antibody. Exemplary anti-PDL1 antibodies include, but are not limited to, Atezolizumab, MPDL3280A, Avelumab and Durvalumab.

In some embodiments, the anti-PD1 antibody is administered once every three weeks. In some embodiments, the withholding step comprises withholding the MALT-1 inhibitor for up to 14 days (e.g., 1 day, 2 days, 3 days, 4 days, 5 days, 6 days, 7 days, 8 days, 9 days, 10 days, 11 days, 12 days, 13 days or 14 days).

In some embodiments, the MALT-1 inhibitor is a small molecule. In some embodiments, the MALT-1 inhibitor is MI-2 or an analog thereof, MI-2A1, MI-2A2, MI-2A3, MI-2A4, MI-2A5, MI-2A6, MI-2A7, a pyrazolo pyrimidine derivative, a phenothiazine derivative, a thiazolo-pyridine derivative, or tetrapeptide Z-VRPR-FMK, or a pharmaceutically acceptable salt thereof.

In some embodiments, the MALT-1 inhibitor is mepazine, thioridazine, or promazine, or a pharmaceutically acceptable salt thereof. In some embodiments, the MALT-1 inhibitor is (S)-mepazine or a pharmaceutically acceptable salt thereof.

In any of the embodiments described herein, the cancer to be treated is a carcinoma, a melanoma, a sarcoma, a myeloma, a leukemia, or a lymphoma. In some embodiments, the cancer is melanoma or colon cancer.

In some embodiments, the cancer is a solid tumor. Exemplary solid tumors include, but are not limited to, an Adrenocortical Tumor, an Alveolar Soft Part Sarcoma, a Chondrosarcoma, a Colorectal Carcinoma, a Desmoid Tumors, a Desmoplastic Small Round Cell Tumor, an Endocrine Tumors, an Endodermal Sinus Tumor, an Epithelioid Hemangioendothelioma, a Ewing Sarcoma, a Germ Cell Tumors (Solid Tumor), a Giant Cell Tumor of Bone and Soft Tissue, a Hepatoblastoma, a Hepatocellular Carcinoma, a Melanoma, a Nephroma, a Neuroblastoma, a Non-Rhabdomyosarcoma Soft Tissue Sarcoma (NRSTS), an Osteosarcoma, a Paraspinal Sarcoma, a Renal Cell Carcinoma, a Retinoblastoma, a Rhabdomyosarcoma, a Synovial Sarcoma, or a Wilms Tumor.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology of the following.

### BRIEF DESCRIPTION OF THE FIGURES

FIG. 1 is a graph showing interval versus continuous mepazine treatment in combination with anti-PD-1 checkpoint inhibition therapy for melanoma.

FIG. 2 is a graph showing tumor volume over time in mice administered (S)-mepazine in a ×3 dose regimen or a ×21 dose regimen, compared to anti-PD1 alone or vehicle.

FIG. 3 is a graph showing tumor volume over time in mice administered (S)-mepazine in a ×7 dose regimen or a ×21 dose regimen, compared to anti-PD1 alone or vehicle.

FIG. 4 is a graph showing tumor volume over time in mice administered (S)-mepazine daily (QD) or 5 days on/5 days off, each in combo with an anti-PD-1, compared to anti-PD-1, alone.

### DETAILED DESCRIPTION

The present disclosure provides a method of treating cancer comprising administering a checkpoint inhibitor; administering a first dose of a MALT-1 inhibitor for a first period of time (e.g., up to 7 days); withholding administration of the MALT-1 inhibitor for a second period of time (e.g., up to 14 days); and administering a second dose of the MALT-1 inhibitor. Intermittent dosing of the MALT-1 inhibitor results in an attenuation of the anti-tumor effect of immune cells other than Treg, including cytotoxic T cells (CTL) and natural killer (NK) cells. It is contemplated that short term administration of a MALT-1 inhibitor will destabilize Treg and cause them to secrete IFN-γ, which in turn will inflame tumors and recruit additional anti-tumor effector cells (e.g., CTL, NK cells). MALT1 inhibition attenuates the function of CTL and NK cells, but the drug holiday, after Treg destabilization is achieved, leaves the function of CTL and NK cell unimpaired and thereby can maximize the effect of MALT1 inhibition through Treg destabilization. After a few days of drug holiday (i.e., withholding administration of the MALT-1 inhibitor), newly recruited Treg are again destabilized through another dosing of MALT1 inhibitors.

### MALT-1 Inhibitors

In some embodiments, the MALT-1 inhibitor inhibits MALT-1 paracaspase activity. In some embodiments, the inhibitor of MALT-1 paracaspase activity is MALT-1 Inhibitor-2 (MI-2, chemical name: 2-Chloro-N-[4-[5-(3,4-dichlorophenyl)-3-(2-methoxyethoxy)-1H-1,2,4-triazol-1-yl]phenylacetamide). MI-2 directly binds MALT-1 and irreversibly suppresses the protease function of MALT-1, and is commercially available from Tocris; Cat No. 4848; Minneapolis, Minn.

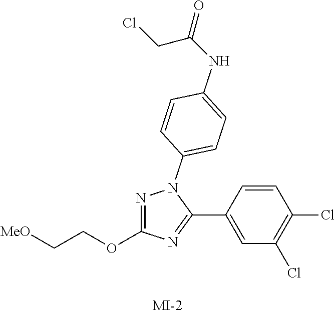

In some embodiments, the inhibitor of MALT-1 paracaspase activity is an analog of MI-2. Analogs of MI-2 (MI-2A1, MI-2A2, MI-2A3, MI-2A4, MI-2A5, MI-2A6, and MI-2A7) have been identified as having anti-MALT-1 paracaspase activity, and are further described in e.g., Fontan, L, et al. Cancer Cell. 2012 Dec. 11; 22(6): 812-824, and Xin B T, et al. Bioorganic and Medicinal Chemistry 24, 2016: 3312-3329, which are incorporated herein by reference in their entireties.

In some cases, the analogs of MI-2 are disclosed in WO 2014/074815, the disclosure of which is incorporated by reference in its entirety. In some cases, the MALT-1 inhibitor is a compound as disclosed in WO 2014/074815, the disclosure of which is incorporated by reference in its entirety. In some cases, the MALT1 inhibitor has a structure of

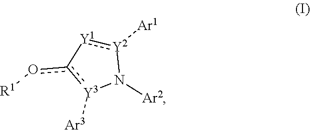

where a dashed bond indicates that a bond can be present or absent; when a double bond is present between Y¹ and Y², Y¹ is N or CR, Y² is C, and Ar¹ is present; when a single bond is present between Y¹ and Y², Y¹ is CR₂, Y² is O or S, and Ar¹ is absent, and each independently selected R is H or (CI-C6)alkyl; R¹ is alkyl, alkoxyalkyl, or arylalkyl, wherein any alkyl, alkoxyalkyl, or arylalkyl, can be mono- or independently multi-substituted with halo or (C1-C6)alkoxy, provided that when a double bond is present between the oxygen atom and the ring comprising Y³, R¹ is absent and Ar³ is present, and when a single bond is present between the oxygen atom and the ring, R¹ is present, a double bond between Y and the carbon atom bearing the oxygen atom is present, and Ar³ is absent; Ar¹ is phenyl substituted with 1-3 J¹ groups; J¹ is halo or (C 1-C6)alkoxy; Ar² is phenyl substituted with 1-3 J² groups; J² is a group of formula —N(R)C(O)—R² and R² is alkyl, aryl, or arylamino, wherein any alkyl, aryl, or arylamino is substituted with 0-2 halo, nitro, or (C1-C6)alkoxy groups; Ar³ is phenyl substituted with 1-3 J³ groups; and J³ is halo or (C 1-C6)alkoxy. In some cases, the compound is

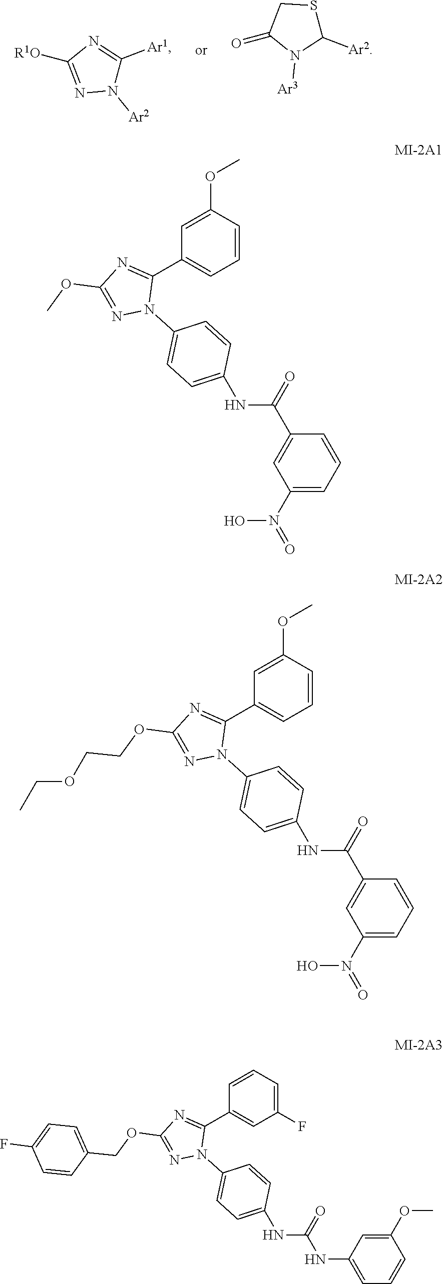

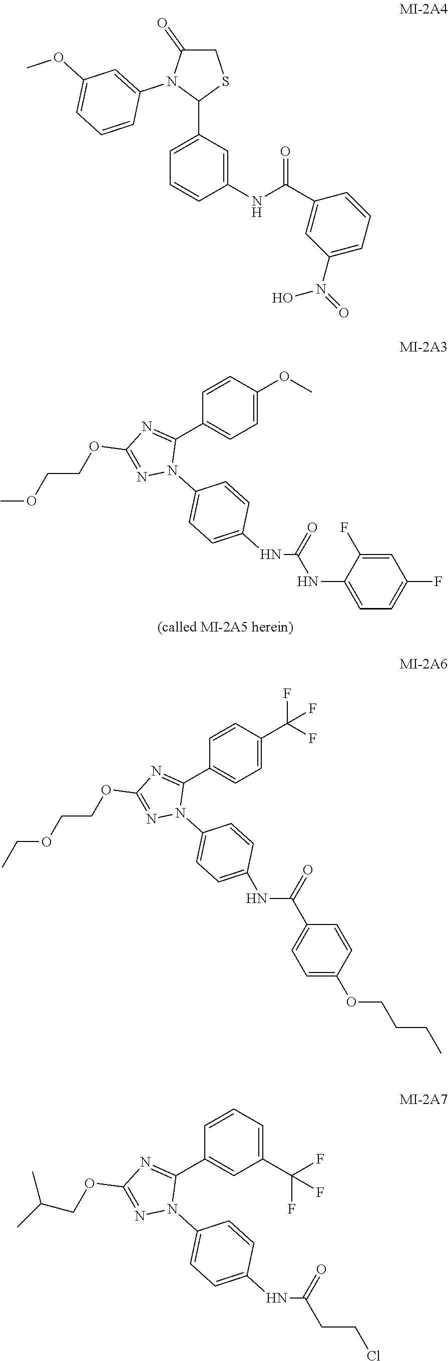

In other embodiments, the inhibitor of MALT-1 paracaspase activity is a pyrazolo pyrimidine derivative. The inhibitory MALT-1 action of the family of pyrazolo pyrimidine derivatives is further described in, e.g., U.S. patent application Ser. No. 15/312,321 or WO 2015/181747, each of which is incorporated herein by reference in its entirety. The pyrazolo pyrimidine derivative can have a structure of Formula (1) as disclosed in WO 2015/181747

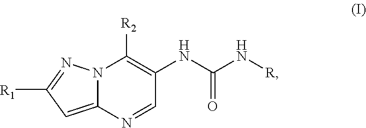

wherein
R₁ is halogen, cyano, or C₁-C₃alkyl optionally substituted by halogen;
R₂ is C₁-C₆ alkyl optionally substituted one or more times by C₁-C₆ alkyl, CrC₆ alkenyl, hydroxyl, N,N-di-CrC₆ alkylamino, N-mono-CrC₆ alkylamino, O-Rg, Rg, phenyl, or by C₁-C₆ alkoxy wherein said alkoxy again may optionally be substituted by C₁-C₆ alkoxy, N,N-di-CrC₆ alkylamino, Rg or phenyl; C₃-C₆cycloalkyl optionally substituted by C₁-C₆ alkyl, N,N-di-CrC₆ alkylamino or Ci-C₆ alkoxy-Ci-C₆ alkyl, and/or two of said optional substituents together with the atoms to which they are bound may form an annulated or spirocyclic 4-6 membered saturated heterocyclic ring comprising 1-20 atoms; phenyl optionally substituted by C₁-C₆ alkoxy; a 5-6 membered heteroaryl ring having 1 to 3 heteroatoms selected from N and O said ring being optionally substituted by C₁-C₆ alkyl which may be optionally substituted by amino or hydroxy; Rg; or N,N-di-CrC₆ alkyl amino carbonyl; wherein
Rg is a 5-6 membered heterocyclic ring having 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is phenyl independently substituted two or more times by Ra, 2-pyridyl independently substituted one or more times by Rb, 3-pyridyl independently substituted one or more times by Rc, or 4-pyridyl independently substituted one or more times by Rd; wherein Ra independently from each other is halogen; cyano; —COOCrC₆alkyl; C₁-C₆alkoxy; C₁-C₆ alkyl optionally substituted by halogen or a 5-6 membered heterocyclyl ring having 1 to 2 heteroatoms selected from N and O which ring is optionally substituted by C₁-C₆ alkyl; a 5-6 membered heteroaryl ring having 1 to 3 heteroatoms selected from N and O said ring being optionally substituted by amino, C₁-C₆ alkyl optionally substituted by amino or hydroxy, or by N-mono- or N,N-di-CrC₆ alkylamino carbonyl; and/or
two Ra together with the ring atoms to which they are bound may form a 5 to 6 membered heterocyclic or heteroaromatic ring having 1 to 2 N atoms, any such ring being optionally substituted by C₁-C₆alkyl or oxo;
Rb, Rc and Rd independently from each other are halogen; oxo; hydroxy; cyano; C₁-C₆ alkoxy optionally substituted by halogen; C₁-C₆ alkoxy carbonyl; phenyl; N,N-di-CrC₆ alkyl amino; C₁-C₆ alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N-d-C₆ alkylamino carbonyl; O—Rh; or Rh; wherein
Rh is a 5-6 membered heterocyclyl ring having 1 to 4 heteroatoms selected from N, O and S said ring being optionally substituted by C₁-C₆ alkyl, hydroxyl or oxo.

Pyrazolo pyrimidine derivatives include, but are not limited to, Zaleplon™, Indiplon™, Ocinaplon, Divaplon, and Lorediplon. Pyrazolo pyrimidine derivatives are a series of isomeric heterocyclic chemical compounds with the molecular formula C₆H₅N₃. They form the central core of various complex chemical compounds including, for example, some pharmaceuticals and pesticides. One isomer of pyrazolo pyrimidines, known as pyrazolo[1,5-a]pyrimidine, is the basis for a class of sedative and anxiolytic drugs related (in terms of their effect) to benzodiazepines. In one embodiment, the inhibitor of MALT-1 paracaspase activity comprises a chemical structure comprising pyrazolo[1,5-a]pyrimidine.

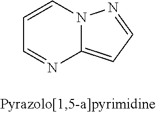

In some cases, the MALT-1 inhibitor is a pyrazolo pyrimidine derivative selected from (S)-1-(5-cyanopyridin-3-yl)-3-(7-(1-methoxyethyl)-2-methylpyrazolo [1,5-a]pyrimidin-6-yl)urea; (S)-1-(2-(difluoromethyl)pyridin-4-yl)-3-(2-fluoro-7-(1-methoxyethyl) pyrazolo[1,5-a]pyrimidin-6-yl)urea; (S)-1-(2-chloro-7-(1-methoxyethyl)pyrazolo[1,5-a]pyrimidin-6-yl)-3-(2-(trifluoromethyl)pyridin-4-yl)urea; 1-(5-chloro-6-(2H-1,2,3-triazol-2-yl)pyridin-3-yl)-3-(2-chloro-7-isopropylpyrazolo[1,5-a]pyrimidin-6-yl)urea; (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea; (S)-1-(5-cyano-6-methoxypyridin-3-yl)-3-(2-fluoro-7-(1-methoxyethyl) pyrazolo[1,5-a]pyrimidin-6-yl)urea; (S)-1-(6-(2H-1,2,3-triazol-2-yl)-5-(trifluoromethyl)pyridin-3-yl)-3-(2-chloro-7-(1-(2-methoxyethoxy)ethyl)pyrazolo[1,5-a]pyrimidin-6-yl)urea; (S)-1-(6-(2H-1,2,3-triazol-2-yl)-5-(trifluoromethyl)pyridin-3-yl)-3-(2-chloro-7-(1-methoxy-2-methyl-propyl)pyrazolo[1,5-a]pyrimidin-6-yl)urea; 1-(2-chloro-7-(1-(methoxymethyl) cyclopropyl)pyrazolo[1,5-a]pyrimidin-6-yl)-3-(5-cyanopyridin-3-yl)urea; 1-(5-chloro-6-(2H-1,2,3-triazol-2-yl)pyridin-3-yl)-3-(2-chloro-7-((1R,2S)-1,2-dimethoxypropyl) pyrazolo[1,5-a]pyrimidin-6-yl)urea; (S)-1-(2-chloro-7-(1-methoxyethyl)pyrazolo[1,5-a]pyrimidin-6-yl)-3-(5-cyano-6-(2H-1,2,3-triazol-2-yl)pyridin-3-yl)urea; (S)-1-(5-cyanopyridin-3-yl)-3-(2-fluoro-7-(1-methoxyethyl)pyrazolo[1,5-a]pyrimidin-6-yl) urea; 1-(7-((S)-1-(((R)-1-acetylpyrrolidin-3-yl)oxy)ethyl)-2-chloropyrazolo[1,5-a]pyrimidin-6-yl)-3-(5-chloro-6-(2H-1,2,3-triazol-2-yl)pyridin-3-yl)urea; (S)-1-(5-chloro-6-(2H-1,2,3-triazol-2-yl)pyridin-3-yl)-3-(2-fluoro-7-(1-methoxy-2-methylpropyl)-pyrazolo[1,5-a]pyrimidin-6-yl)urea; (S)-1-(5-cyano-6-(2H-1,2,3-triazol-2-yl)pyridin-3-yl)-3-(7-(1-methoxy-2-methylpropyl)-2-methylpyrazolo[1,5-a]pyrimidin-6-yl)urea; (S)-1-(2-chloro-7-(1-methoxyethyl)pyrazolo[1,5-a]pyrimidin-6-yl)-3-(5-cyano-6-methoxypyridin-3-yl)urea; 1-(2-fluoro-7-((S)-1-methoxyethyl)pyrazolo[1,5-a]pyrimidin-6-yl)-3-(2-(1-hydroxyethyl)-6-(trifluoromethyl)pyridin-4-yl)urea; (S)-1-(5-cyano-6-(2H-1,2,3-triazol-2-yl)pyridin-3-yl)-3-(2-fluoro-7-(1-methoxyethyl)pyrazolo[1,5-a]pyrimidin-6-yl)urea; 1-(2-chloro-7-(1,2-dimethoxyethyl)pyrazolo[1,5-a]pyrimidin-6-yl)-3-(5-cyano-6-(2H-1,2,3-triazol-2-yl)pyridin-3-yl)urea; 1-(2-chloro-7-((S)-1-methoxyethyl)pyrazolo[1,5-a]pyrimidin-6-yl)-3-(2-(2,2,2-trifluoro-1-hydroxy-ethyl)pyridin-4-yl)urea; (S)-1-(5-chloro-2-(2-methoxyethoxy)pyridin-3-yl)-3-(2-chloro-7-(1-methoxyethyl)-pyrazolo[1,5-a]-pyrimidin-6-yl)urea; (S)-1-(5-cyano-6-methoxypyridin-3-yl)-3-(7-(1-methoxy-2-methylpropyl)-2-methylpyrazolo[1,5-a]-pyrimidin-6-yl)urea; (S)-1-(2-cyanopyridin-4-yl)-3-(2-fluoro-7-(1-methoxyethyl)pyrazolo[1,5-a]pyrimidin-6-yl)urea; (S)-1-(5-cyano-6-methoxypyridin-3-yl)-3-(7-(1-methoxyethyl)-2-methylpyrazolo[1,5-a]pyrimidin-6-yl)urea; 1-(2-chloro-7-((1R,2S)-1,2-dimethoxypropyl)pyrazolo[1,5-a]pyrimidin-6-yl)-3-(5-cyano-6-(2H-1,2,3-triazol-2-yl)pyridin-3-yl)urea; 1-(7-((S)-1-(((S)-1-acetylpyrrolidin-3-yl)oxy)ethyl)-2-chloropyrazolo[1,5-a]pyrimidin-6-yl)-3-(5-cyano-6-methoxypyridin-3-yl)urea; (S)-1-(5-cyano-6-(2H-1,2,3-triazol-2-yl)pyridin-3-yl)-3-(7-(1-methoxyethyl)-2-methylpyrazolo[1,5-a]pyrimidin-6-yl)urea; (S)-6-chloro-4-(3-(2-chloro-7-(1-methoxyethyl)pyrazolo[1,5-a]pyrimidin-6-yl)ureido)-N,N-dimethylpicolinamide; (S)-1-(5-(difluoro-methyl)pyridin-3-yl)-3-(2-fluoro-7-(1-methoxyethyl)-pyrazolo[1,5-a]pyrimidin-6-yl)urea; (S)-1-(2-fluoro-7-(1-methoxyethyl)pyrazolo[1,5-a]pyrimidin-6-yl)-3-(5-(trifluoro-methyl) pyridin-3-yl)urea; (S)-3-chloro-5-(3-(2-chloro-7-(1-methoxyethyl)pyrazolo[1,5-a]pyrimidin-6-yl)ureido)-N,N-dimethylpicolinamide; (S)-1-(5-chloro-pyridin-3-yl)-3-(2-fluoro-7-(1-methoxyethyl)pyrazolo[1,5-a]pyrimidin-6-yl)urea; (S)-1-(5-chloro-6-(pyrrolidine-1-carbonyl)pyridin-3-yl)-3-(2-chloro-7-(1-methoxyethyl)pyrazolo-[1,5-a]pyrimidin-6-yl)urea (S)-3-chloro-5-(3-(2-chloro-7-(1-methoxyethyl)pyrazolo[1,5-a]pyrimidin-6-yl)ureido)-N-methylpicolinamide; (S)-1-(2-chloro-7-(1-methoxyethyl)pyrazolo[1,5-a]pyrimidin-6-yl)-3-(5-chloropyridin-3-yl)urea; (S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea; (S)-1-(5-cyanopyridin-3-yl)-3-(7-(1-hydroxyethyl)-2-methylpyrazolo [1,5-a]pyrimidin-6-yl)urea; (S)-1-(2-(difluoromethyl)pyridin-4-yl)-3-(2-fluoro-7-(1-hydroxyethyl) pyrazolo[1,5-a]pyrimidin-6-yl)urea; 1-(2-((S)-2-aminopropoxy)-5-chloropyridin-3-yl)-3-(2-chloro-7-((S)-1-methoxyethyl)pyrazolo[1,5-a]pyrimidin-6-yl)urea; S)-2-(difluoromethyl)-4-(3-(2-fluoro-7-(1-methoxyethyl)pyrazolo[1,5-a]pyrimidin-6-yl)ureido)pyridine 1-oxide; 1-(2-chloro-7-((1R,2S)-1,2-dimethoxypropyl)pyrazolo[1,5-a]pyrimidin-6-yl)-3-(5-cyano-6-methoxypyridin-3-yl)urea; 1-(2-chloro-7-(1-(methoxymethyl)cyclopropyl)pyrazolo[1,5-a]pyrimidin-6-yl)-3-(2-cyanopyridin-4-yl)urea; and (S)-3-chloro-5-(3-(2-fluoro-7-(1-methoxyethyl)pyrazolo[1,5-a]pyrimidin-6-yl)ureido)picolinamide.

In some cases, the pyrazolo pyrimidine MALT-1 inhibitor compound is as disclosed in International Publication No. WO 2017/081641, the disclosure of which is incorporated by reference in its entirety. The compound can have a structure of

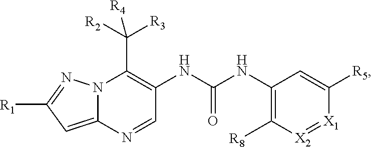

where R1 is fluoro, chloro, methyl or cyano; R2 and R3 are independently from each other C₁-C₆ alkoxy optionally substituted by C₁-C₆ alkoxy; C₁-C₆ alkyl optionally substituted by halogen or C₁-C₆ alkoxy; amino optionally substituted by C₁-C₆ alkyl; phthalimido; or hydroxy optionally substituted by a 5 or 6 membered heterocyclic ring comprising a nitrogen or oxygen heteroatom wherein said ring is optionally substituted by C₁-C₃ alkyl carbonyl; or R2 and R3 together with carbon atom to which they are attached form a 3-5 membered carbocyclic ring or heterocyclic ring comprising 1 heteroatom selected from N and O; R4 is hydrogen; C₁-C₆ alkyl optionally substituted by C₁—C alkoxy; X1 is N, N—O, or CR⁶; X₂ is N or CR7; R5 is chloro; cyano; or C₁-C₆ alkyl optionally substituted by halogen and/or hydroxy; R6 is hydrogen; oxo; methoxy; 1,2,3-triazole-2-yl; or aminocarbonyl substituted at the nitrogen atom by R9 and R10; R7 is hydrogen; C₁-C₆ alkyl optionally substituted by halogen and/or hydroxy; or N,N-dimethylaminocarbonyl; R8 is hydrogen; C₁-C₆ alkoxy optionally substituted by methoxy or amino; R9 and 10 are independently of each other hydrogen; C₁-C₆ alkyl optionally substituted by C₁-C₆ alkoxy, N-mono-C₁-C₆ alkyl amino, or N,N-di-C₁-C₆ alkyl amino; or R9 and 10 together with the nitrogen atom to which they are attached form a 5-7 membered heterocyclic ring having one, two or three ring hetero atoms selected from the group consisting of oxygen, nitrogen and sulfur, that ring being optionally substituted by C₁—C alkyl, hydroxy or oxo; with the proviso that X1 and X₂ must not be N at the same time, or X1 must not be N—O when X₂ is N. In some cases, the compound is selected from (S)-1-(2-(difluoromethyl)pyridin-4-yl)-3-(2-fluoro-7-(1-methoxyethyl) pyrazolo[1,5-a]pyrimidin-6-yl)urea; (S)-1-(2-chloro-7-(1-methoxyethyl)pyrazolo[1,5-a]pyrimidin-6-yl)-3-(2-(trifluoromethyl)pyridin-4-yl)urea; 1-(5-chloro-6-(2H-1,2,3-triazol-2-yl)pyridin-3-yl)-3-(2-chloro-7-isopropylpyrazolo[1,5-a]pyrimidin-6-yl)urea; (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea; (S)-1-(5-cyano-6-methoxypyridin-3-yl)-3-(2-fluoro-7-(1-methoxyethyl) pyrazolo[1,5-a]pyrimidin-6-yl)urea; (S)-1-(6-(2H-1,2,3-triazol-2-yl)-5-(trifluoromethyl)pyridin-3-yl)-3-(2-chloro-7-(1-(2-methoxyethoxy) ethyl)pyrazolo[1,5-a]pyrimidin-6-yl)urea; (S)-1-(6-(2H-1,2,3-triazol-2-yl)-5-(trifluoromethyl)pyridin-3-yl)-3-(2-chloro-7-(1-methoxy-2-methyl-propyl)pyrazolo[1,5-a]pyrimidin-6-yl)urea; 1-(2-chloro-7-(1-(methoxymethyl)cyclopropyl)pyrazolo[1,5-a]pyrimidin-6-yl)-3-(5-cyanopyridin-3-yl)urea; 1-(5-chloro-6-(2H-1,2,3-triazol-2-yl)pyridin-3-yl)-3-(2-chloro-7-((1R,2S)-1,2-dimethoxypropyl) pyrazolo[1,5-a]pyrimidin-6-yl)urea; (S)-1-(2-chloro-7-(1-methoxyethyl)pyrazolo[1,5-a]pyrimidin-6-yl)-3-(5-cyano-6-(2H-1,2,3-triazol-2-yl)pyridin-3-yl)urea; (S)-1-(5-cyanopyridin-3-yl)-3-(2-fluoro-7-(1-methoxyethyl)pyrazolo[1,5-a]pyrimidin-6-yl)urea; 1-(7-((S)-1-(((R)-1-acetylpyrrolidin-3-yl)oxy)ethyl)-2-chloropyrazolo[1,5-a]pyrimidin-6-yl)-3-(5-chloro-6-(2H-1,2,3-triazol-2-yl)pyridin-3-yl)urea; (S)-1-(5-chloro-6-(2H-1,2,3-triazol-2-yl)pyridin-3-yl)-3-(2-fluoro-7-(1-methoxy-2-methylpropyl)-pyrazolo[1,5-a]pyrimidin-6-yl)urea; (S)-1-(5-cyano-6-(2H-1,2,3-triazol-2-yl)pyridin-3-yl)-3-(7-(1-methoxy-2-methylpropyl)-2-methylpyrazolo[1,5-a]pyrimidin-6-yl)urea; (S)-1-(2-chloro-7-(1-methoxyethyl)pyrazolo[1,5-a]pyrimidin-6-yl)-3-(5-cyano-6-methoxypyridin-3-yl)urea; 1-(2-fluoro-7-((S)-1-methoxyethyl)pyrazolo[1,5-a]pyrimidin-6-yl)-3-(2-(1-hydroxyethyl)-6-(trifluoromethyl)pyridin-4-yl)urea; (S)-1-(5-cyano-6-(2H-1,2,3-triazol-2-yl)pyridin-3-yl)-3-(2-fluoro-7-(1-methoxyethyl)pyrazolo[1,5-a]pyrimidin-6-yl)urea; 1-(2-chloro-7-(1,2-dimethoxyethyl)pyrazolo[1,5-a]pyrimidin-6-yl)-3-(5-cyano-6-(2H-1-triazol-2-yl)pyridin-3-yl)urea; 1-(2-chloro-7-((S)-1-methoxyethyl)pyrazolo[1,5-a]pyrimidin-6-yl)-3-(2-(2,2,2-trifluoro-1-hydroxy-ethyl)pyridin-4-yl)urea; (S)-1-(5-chloro-2-(2-methoxyethoxy)pyridin-3-yl)-3-(2-chloro-7-(1-methoxyethyl)-pyrazolo[1,5-a]-pyrimidin-6-yl)urea; (S)-1-(5-cyano-6-methoxypyridin-3-yl)-3-(7-(1-methoxy-2-methylpropyl)-2-methylpyrazolo[1,5-a]-pyrimidin-6-yl)urea; (S)-1-(2-cyanopyridin-4-yl)-3-(2-fluoro-7-(1-methoxyethyl)pyrazolo[1,5-a]pyrimidin-6-yl)urea; (S)-1-(5-cyano-6-methoxypyridin-3-yl)-3-(7-(1-methoxyethyl)-2-methylpyrazolo[1,5-a]pyrimidin-6-yl)urea; 1-(2-chloro-7-((1R,2S)-1,2-dimethoxypropyl)pyrazolo[1,5-a]pyrimidin-6-yl)-3-(5-cyano-6-(2H-1,2,3-triazol-2-yl)pyridin-3-yl)urea; 1-(7-((S)-1-(((S)-1-acetylpyrrolidin-3-yl)oxy)ethyl)-2-chloropyrazolo[1,5-a]pyrimidin-6-yl)-3-(5-cyano-6-methoxypyridin-3-yl)urea; (S)-1-(5-cyano-6-(2H-1,2,3-triazol-2-yl)pyridin-3-yl)-3-(7-(1-methoxyethyl)-2-methylpyrazolo[1,5-a]pyrimidin-6-yl)urea; (S)-6-chloro-4-(3-(2-chloro-7-(1-methoxyethyl)pyrazolo[1,5-a]pyrimidin-6-yl)ureido)-N,N-dimethylpicolinamide; (S)-1-(5-(difluoro-methyl)pyridin-3-yl)-3-(2-fluoro-7-(1-methoxyethyl)-pyrazolo[1,5-a]pyrimidin-6-yl)urea; (S)-1-(2-fluoro-7-(1-methoxyethyl)pyrazolo[1,5-a]pyrimidin-6-yl)-3-(5-(trifluoro-methyl)pyridin-3-yl)urea; (S)-3-chloro-5-(3-(2-chloro-7-(1-methoxyethyl)pyrazolo[1,5-a]pyrimidin-6-yl)ureido)-N,N-dimethylpicolinamide; (S)-1-(5-chloro-pyridin-3-yl)-3-(2-fluoro-7-(1-methoxyethyl)pyrazolo[1,5-a]pyrimidin-6-yl)urea; (S)-1-(5-chloro-6-(pyrrolidine-1-carbonyl)pyridin-3-yl)-3-(2-chloro-7-(1-methoxyethyl)pyrazolo-[1,5-a]pyrimidin-6-yl)urea; (S)-3-chloro-5-(3-(2-chloro-7-(1-methoxyethyl)pyrazolo[1,5-a]pyrimidin-6-yl)ureido)-N-methylpicolinamide; (S)-1-(2-chloro-7-(1-methoxyethyl)pyrazolo[1,5-a]pyrimidin-6-yl)-3-(5-chloropyridin-3-yl)urea; (S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea; (S)-1-(5-cyanopyridin-3-yl)-3-(7-(1-hydroxyethyl)-2-methylpyrazolo [1,5-a]pyrimidin-6-yl)urea; (S)-1-(2-(difluoromethyl)pyridin-4-yl)-3-(2-fluoro-7-(1-hydroxyethyl) pyrazolo[1,5-a]pyrimidin-6-yl)urea; 1-(2-((S)-2-aminopropoxy)-5-chloropyridin-3-yl)-3-(2-chloro-7-((S)-1-methoxyethyl)pyrazolo[1,5-a]pyrimidin-6-yl)urea; (S)-2-(difluoromethyl)-4-(3-(2-fluoro-7-(1-methoxyethyl)pyrazolo[1,5-a]pyrimidin-6-yl)ureido)pyridine 1-oxide; 1-(2-chloro-7-((1R,2S)-1,2-dimethoxypropyl)pyrazolo[1,5-a]pyrimidin-6-yl)-3-(5-cyano-6-methoxypyridin-3-yl)urea; 1-(2-chloro-7-(1-(methoxymethyl)cyclopropyl)pyrazolo[1,5-a]pyrimidin-6-yl)-3-(2-cyanopyridin-4-yl)urea; and (S)-3-chloro-5-(3-(2-fluoro-7-(1-methoxyethyl)pyrazolo[1,5-a]pyrimidin-6-yl)ureido)picolinamide.

In some cases, the MALT-1 inhibitor is compound as disclosed in International Publication No. WO 2018/085247, the disclosure of which is incorporated by reference in its entirety. In some cases, the compound has a structure

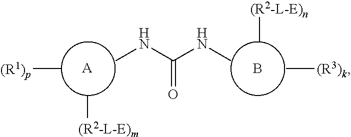

wherein A is a fused bicyclic heteroaryl ring; B is phenyl or pyridinyl; each occurrence of R¹ and R³ is, independently, hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, OC(═O)R^{A}, —OC(═O)OR^{A}, ˜ OC(═O)N(R^{A})₂, or a nitrogen protecting group when attached to a nitrogen atom; R is substituted or unsubstituted alkylene, substituted or unsubstituted heterocyclylene, substituted or unsubstituted arylene, substituted or unsubstituted heteroarylene, substituted or unsubstituted heteroalkylene, substituted or unsubstituted alkylheteroarylene, substituted or unsubstituted heteroarylalkylene, —O—, —N(R^{A}), —C(═O)—, —C(═O)O—, —C(═O)NR^{A}—, —NR^{A}C(═O)—, —NR^{A}C(═O)O—, —NR^{A}C(═O)N(R^{A}), —OC(═O)—, —OC(═O)O—, or —OC(═O)N(R^{A})—; each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclic ring; L is substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, —O—, —N(R^{A})—, —S—, —C(═O)—, —C(═O)O—, —C(═O)NR^{A}—, —NR^{A}C(═O)—, —NR^{A}C(═O)R^{A}, —C(═O)R^{A}—, —NR^{A}C(═O)O—, —NR^{A}C(═O)N(R^{A})—, —OC(═O)—, —OC(═O)O—, or —OC(═O)N(R^{A})—, or a combination thereof; E is an E3 ubiquitin ligase binding moiety; m and n are each independently 0 or 1, provided that m+n=1; k is 0, 1, 2, 3, or 4; and p is 0, 1, 2, 3, or 4. In some cases,

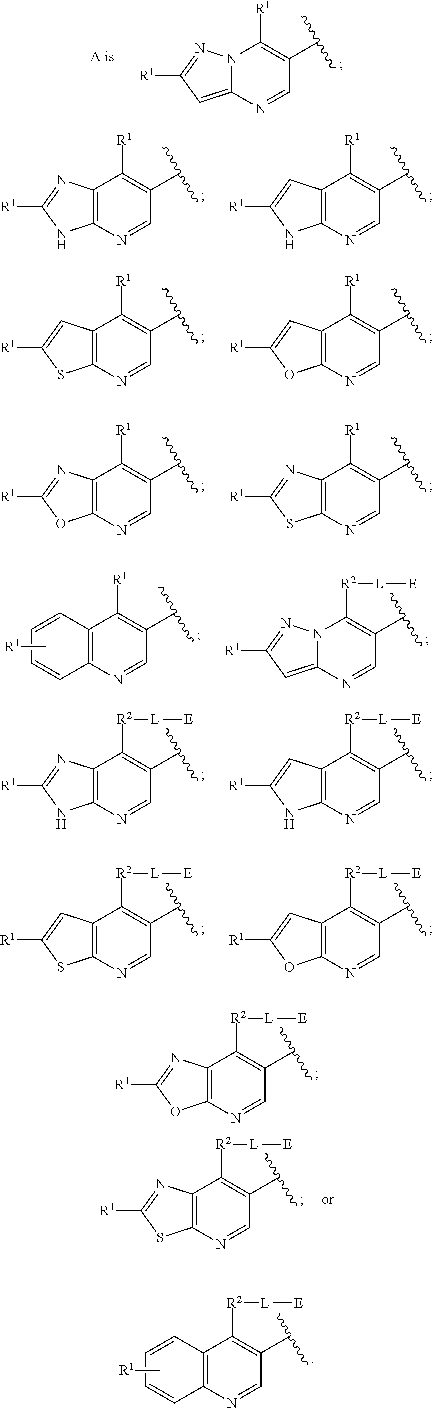

In some cases,

### R²-L- is

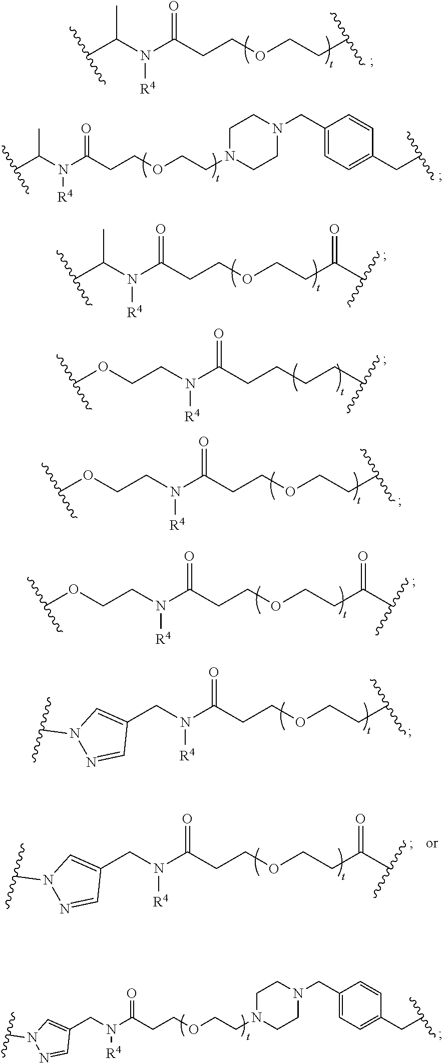

R⁴ is hydrogen or C₁₋₆ alkyl; and t is 0, 1, 2, 3, 4; 5, or 6. In some cases,

E is

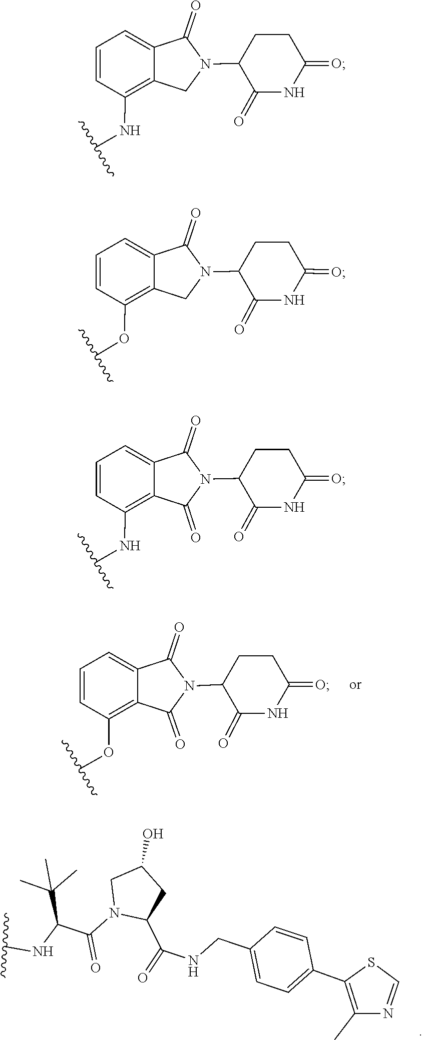 [SMILES: *Nc1cccc2c1CN(C1CCC(=O)NC1=O)C2=O ; *Oc1cccc2c1CN(C1CCC(=O)NC1=O)C2=O ; *Nc1cccc2c1C(=O)N(C1CCC(=O)NC1=O)C2=O ; *Oc1cccc2c1C(=O)N(C1CCC(=O)NC1=O)C2=O ; *N[C@H](C(=O)N1C[C@H](O)C[C@H]1C(=O)NCc1ccc(-c2scnc2C)cc1)C(C)(C)C]

In various cases, the compound has a structure of

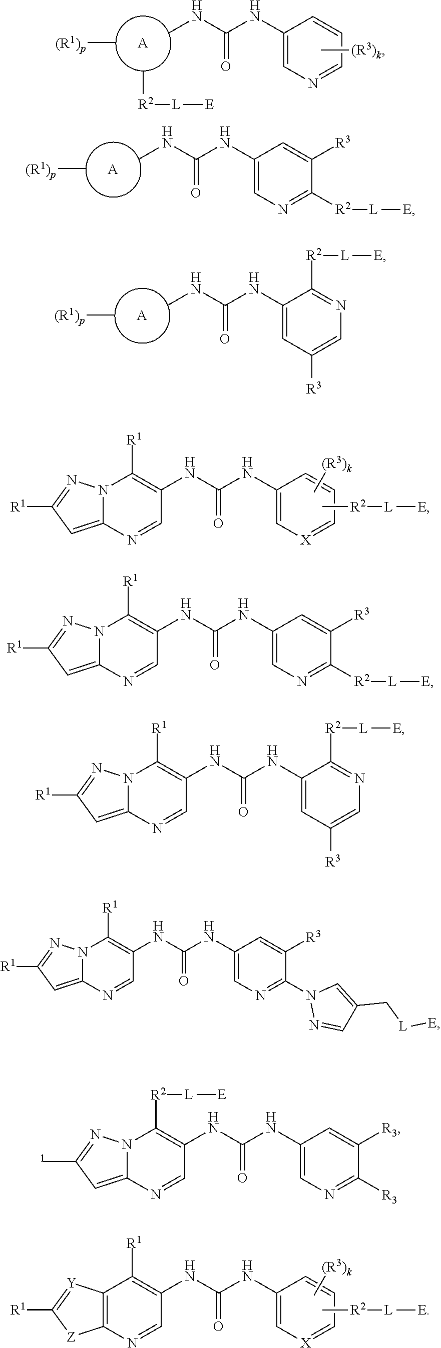

where X is N, CH, or CR³; Y is CH or N, and Z is NH, S, or O;

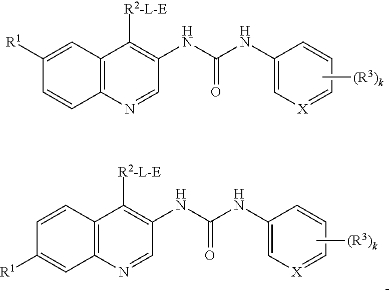

where X is N, CH, or CR³;

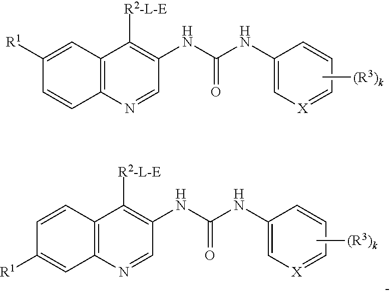

where X is N, CH, or CR³;

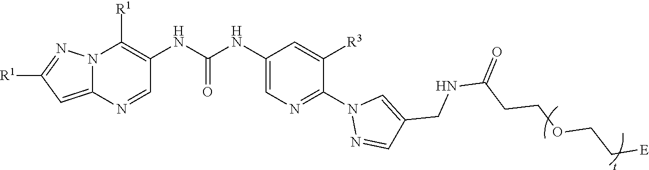

where t is 2 or 4;

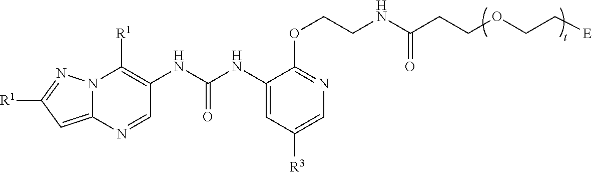

where t is 2 or 4;

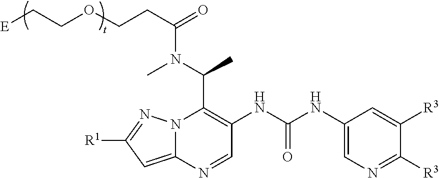

where t is 2 or 4;

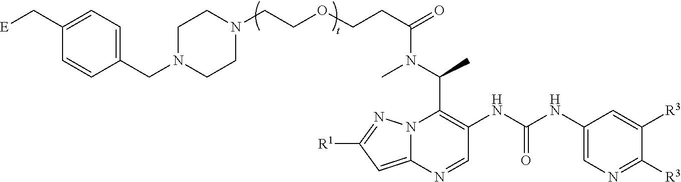

where t is 0, 1, 2, 3, 4, 5, or 6;

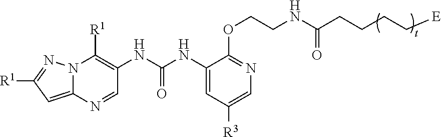

where t is 0, 1, 2, 3, 4, 5, or 6; or

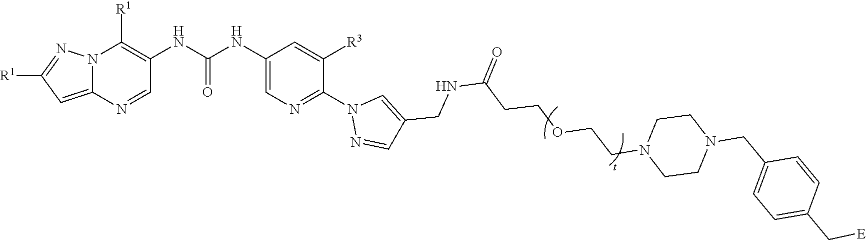

where t is 0, 1, 2, 3, 4, 5, or 6.

In some embodiments, the inhibitor of MALT-1 paracaspase activity is a phenothiazine derivative. Phenothiazine is an organic compound that has the formula S(C₆H₄)₂NH and is related to the thiazine-class of heterocyclic compounds. Phenothiazine has no medicinal use, it is a prototypical lead structure in medicinal chemistry and derivatives of Phenothiazine are widely used. Derivatives of Phenothiazine comprise the Phenothiazine core structure and include, but are not limited to mepazine, thioridazine, promazine, Chlorpromazine (Thorazine™, Aminazine™, Chlor-PZ™, Klorazine™, Promachlor™, Promapar™, Sonazine™, Chlorprom™, Chlor-Promanyl™, Largactil™), Promazine (Sparine™, Propazine™), Triflupromazine (Clinazine™, Novaflurazine™, Pentazine™, Terfluzine™, Triflurin™, Vesprin™), Mesoridazine (Serentil™), Thiojdazine (Mellaril™, Novoridazine™, Thioril™, Sonapax™), Fluphenazine (Prolixin™, Permitil™, Modecate™, Moditen™), Perphenazine (Trilafon™, Etrafon™, Triavil™, Phenazine™, Etaperazin™), Prochlorperazine (Compazine™, Stemetil™), and Trifluoperazine (Stelazine™, Triphtazine™). In some embodiments, the inhibitor of MALT-1 paracaspase activity comprises a chemical structure comprising Phenothiazine. In some embodiments, the inhibitor of MALT-1 paracaspase activity is mepazine. Mepazine comprises MALT-1 inhibitory action, and is further reviewed in, e.g., Nagel D. et al, Cancer Cell, 2012, which is incorporated herein by reference in its entirety. In some cases, the mepazine is present as (S)-mepazine, or a pharmaceutically acceptable salt thereof. (S)-Mepazine is discussed in detail, e.g., in U.S. Pat. No. 9,718,811, the disclosure of which is incorporated by reference in its entirety.

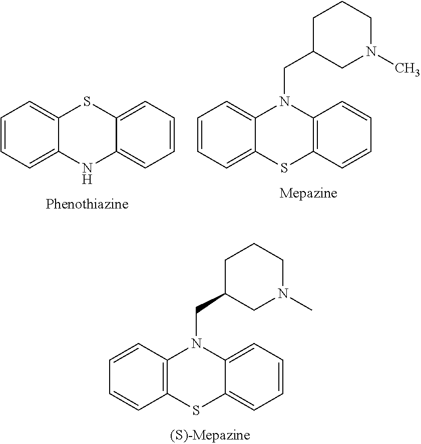

In some embodiments, the MALT-1 inhibitor is a pyrazole derivative, e.g., as disclosed in WO 2018/119036, the disclosure of which is incorporated by reference in its entirety, e.g., having a structure of

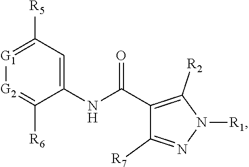

where R₁ is selected from the group consisting of i) naphthalen-I-yl, optionally substituted with a fluoro or amino substituent; and ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluorom ethyl, 1, 1-difluoroethyl, hydroxymethyl, 1-hydroxy ethyl, 1-ethoxy ethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidom ethyl, tetrahydrofuran-2-yl, 3-hydroxy-pyrrolidin-1-yl, pyrrolidin-2-yl, 3-hydroxyazetidinyl, azetidin-3-yl, or azetidin-2-yl; R₂ is selected from the group consisting of C1-4alkyl, 1-methoxy-ethyl, difluoromethyl, fluoro, chloro, bromo, cyano, and trifluoromethyl; G1 is N or C(R₄); G2 is N or C(R₃); such that only one of G1 and G2 are N in any instance; R₃ is independently selected from the group consisting of trifluoromethyl, cyano, C1-4alkyl, fluoro, chloro, bromo, methylcarbonyl, methylthio, methyl sulfinyl, and methanesulfonyl; or, when G1 is N, R3 is further selected from C1-4alkoxycarbonyl; R₄ is selected from the group consisting of i) hydrogen, when G2 is N; ii) Ci-4alkoxy; iii) cyano; iv) cyclopropyloxy; v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b] pyridin-2-yl, 3H-[1,2,3]triazolo[4,5-b]pyridin-3-yl, IH-[1,2,3]triazolo[4,5-c] pyridin-1-yl, wherein the heteroaryl is optionally substituted with one or two substituents independently selected from oxo, C1-4alkyl, carboxy, methoxycarbonyl, aminocarbonyl, hydroxymethyl, aminomethyl, (dimethylamino)methyl, amino, methoxymethyl, trifluoromethyl, amino(C2-4alkyl)amino, or cyano; vi) 1-methyl-piperidin-4-yloxy; vii) 4-methyl-piperazin-1-ylcarbonyl; viii) (4-aminobutyl)aminocarbonyl; ix) (4-amino)butoxy; x) 4-(4-aminobutyl)-piperazin-1-ylcarbonyl; xi) methoxycarbonyl; xii) 5-chloro-6-(methoxycarbonyl)pyridin-3-ylaminocarbonyl; xiii) 1,1-dioxo-isothiazolidin-2-yl; xiv) 3-methyl-2-oxo-2,3-dihydro-1H-imidazol-1-yl; xv) 2-oxopyrrolidin-1-yl; xvi) (E)-(4-aminobut-1-en-1-yl-aminocarbonyl; xvii) difluoromethoxy; and xviii) morpholin-4-ylcarbonyl; R₅ is independently selected from the group consisting of hydrogen, chloro, fluoro, bromo, methoxy, methylsulfonyl, cyano, C1-4alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1, 1-difluoroethyl; or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl; R₆ is hydrogen, C1-4alkyl, fluoro, 2-methoxy-ethoxy, chloro, cyano, or trifluoromethyl; R₇ is hydrogen or fluoro. In some cases, the MALT-1 inhibitor is a compound as listed in Table 1 (compound 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, or 450) at pages 40-133 of WO 2018/119036.

In various cases, the MALT-1 inhibitor is a compound as disclosed in International Publication No. WO 2018/021520, the disclosure of which is incorporated by reference in its entirety herein.

In other embodiments, the inhibitor of MALT-1 paracaspase activity is tetrapeptide Z-VRPR-FMK (Z-VRPR-FMK; C₃₁H₄₉FN₁₀O₆). Z-VRPR-FMK is a selective MALT-1 inhibitor MALT-1's proteolytic activity of the paracaspase.

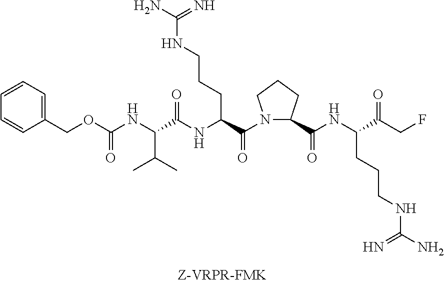 [SMILES: CC(C)[C@H](NC(=O)OCc1ccccc1)C(=O)N[C@@H](CCCNC(=N)N)C(=O)N1CCC[C@H]1C(=O)N[C@@H](CCCNC(=N)N)C(=O)CF]

Other MALT-1 inhibitors contemplated for use in the disclosed methods include thiazolo-pyridines, e.g., those as disclosed in International Publication No. WO 2018/020474, the disclosure of which is incorporated by reference in its entirety. In some cases, the thiazolo-pyridine has a structure of

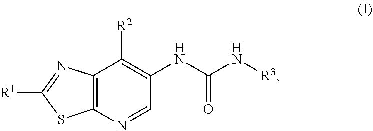

where R¹ is selected from hydrogen, halogen, cyano, substituted or unsubstituted alkyl, and cycloalkyl; R² is selected from—a) alkyl or alkyl substituted with 1 to 4 substituents independently selected from oxo (═O), halogen, cyano, cycloalkyl, substituted or unsubstituted aryl, heteroaryl, substituted or unsubstituted heterocyclyl, —OR⁴, —C(═O)OH, —SO₂(alkyl), —C(═O)O(alkyl), —NR⁵R^{5A}, —NR⁵C(═O)R⁶, C(═O)R⁶, and C(═O)NR⁵R^{5A}; b) cycloakyl or cycloakyl substituted with 1 to 4 substituents independently selected from halogen, cyano, substituted or unsubstituted alkyl, —OR⁴, —C(═O)OH, —C(═O)O(alkyl), C(═O)R⁶, and C(═O)NR⁵R^{5A}; c) cycloalkenyl, d) cyano, e) substituted or unsubstituted aryl, f) substituted or unsubstituted heteroaryl, g) heterocyclyl or heterocyclyl substituted on either ring carbon atom or a ring nitrogen atom and when it is substituted on ring carbon atom it is substituted with 1 to 4 substituents independently selected from oxo (═O), halogen, cyano, substituted or unsubstituted alkyl, cycloalkyl, —OR⁴, —C(═O)OH, —C(═O)O-alkyl, —C(═O)NR⁵R^{5A}, —NHC(═O)(alkyl), —N(H)R⁵, and —N(alkyl)₂, and when the heterocyle group is substituted on a ring nitrogen, it is substituted with substituents independently selected from alkyl, cycloalkyl, aryl, heteroaryl, SO₂(alkyl), C(═O)R⁶, C(═O)O(alkyl), —C(═O)N(H)R⁵, and —C(═O)N(alkyl)R⁵, and h) —NR^{a}R^{b}, wherein, R^{a} and R^{b} are independently selected from hydrogen, cycloalkyl, and alkyl or alkyl substituted with 1 to 4 substituents independently selected from oxo (═O), halogen, cycloalkyl, —OR⁴, and substituted or unsubstituted aryl; R³ is selected from—a) heteroaryl or heteroaryl substituted with 1 to 4 substituents selected from halogen, cyano, —COOR^{4b}, —OR⁴, substituted or unsubstituted heteroaryl, substituted or unsubstituted alkyl, substituted or unsubstituted heterocyclyl, substituted or unsubstituted aryl, nitro, —SO₂alkyl, —SO₂NH(alkyl), —SO₂NH₂, —SO₂NH(CF₃), —SO₂N(alkyl)₂, —NHSO₂(alkyl), —COR⁶, —CON(H)OH, —CONR⁵R^{5a}, —N(R⁵)COR^{5a}, and —NR⁵R^{5a}, b) aryl or aryl substituted with 1 to 4 substituents selected from halogen, cyano, —COOR^{4b}, —OR^{4a}, substituted or unsubstituted alkyl, substituted or unsubstituted heterocyclyl, substituted or unsubstituted aryl, nitro, —SO₂alkyl, —SO₂NH(alkyl), —SO₂NH₂, —SO₂NH(CF₃), —SO₂N(alkyl)₂, —NHSO₂(alkyl), —COR⁶, —CONR⁵R^{5a}, —CO(NH)OH, —N(R⁵)COR⁵, —NR⁵R^{5a}, and heteroaryl or heteroaryl substituted with 1 to 4 substituents selected from substituted or unsubstituted alkyl, c) heterocyclyl or heterocyclyl substituted with 1 to 4 substituents selected from oxo (═O) and substituted or unsubstituted alkyl, and d)

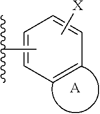

wherein, X is halogen and ring A is a heterocyclic ring containing heteroatom(s) selected from S, O, and N, which is optionally substituted with an oxo (═O) group; R⁴ is selected from hydrogen, cycloalkyl, and substituted or unsubstituted alkyl; R^{4A} is selected from a) hydrogen, alkyl, and cycloalkyl, and b) alkyl substituted with 1 to 4 substituents independently selected from halogen, —O-alkyl, —NR⁵R^{5A}, and substituted or unsubstituted heterocyclyl; R^{4b} is selected from hydrogen and alkyl; R⁵ and R^{5A} are each independently selected from a) hydrogen, alkyl, and cycloalkyl, b) alkyl substituted with O-alkyl, NH₂, and —CONH₂, c) heteroaryl, and d) heterocyclyl substituted with alkyl; and R⁶ is selected from alkyl, heterocyclyl, and cycloalkyl; when an alkyl group is substituted, it is substituted with 1 to 4 substituents independently selected from oxo (═O), halogen, cyano, cycloalkyl, aryl, heteroaryl, heterocyclyl, —OR⁷, —C(═O)OH, —C(═O)O(alkyl), —NR⁸R^{8A}, —NR⁸C(═O)R⁹, and C(═O)NR⁸R^{8A}; when the aryl group is substituted, it is substituted with 1 to 4 substituents independently selected from halogen, nitro, cyano, alkyl, perhaloalkyl, cycloalkyl, heterocyclyl, heteroaryl, —OR⁷, —NR⁸R^{8A}, —NR⁸C(═O)R⁹, C(═O)R⁹, C(═O)NR⁸R^{8A}, —SO₂-alkyl, —C(═O)OH, —C(═O)O-alkyl, and haloalkyl; when the heteroaryl group is substituted, it is substituted with 1 to 4 substituents independently selected from halogen, nitro, cyano, alkyl, haloalkyl, perhaloalkyl, cycloalkyl, heterocyclyl, aryl, heteroaryl, —OR⁷, —NR⁸R^{8a}, —NR⁷C(═O)R⁹, C(═O)R⁹, C(═O)NR⁸NR^{8a}, —SO₂alkyl, —C(═O)OH, and —C(═O)O-alkyl; when the heterocycle group is substituted, it is substituted either on a ring carbon atom or on a ring hetero atom, and when it is substituted on a ring carbon atom, it is substituted with 1 to 4 substituents independently selected from oxo (═O), halogen, cyano, alkyl, cycloalkyl, perhaloalkyl, —OR⁷, C(═O)NR⁸R^{8a}, —C(═O)OH. —C(═O)O-alkyl, —N(H)C(═O)(alkyl), —N(H)R⁸, and —N(alkyl)₂; and when the heterocycle group is substituted on a ring nitrogen, it is substituted with substituents independently selected from alkyl, cycloalkyl, aryl, heteroaryl, —SO₂(alkyl), C(═O)R⁹, and C(═O)O(alkyl); when the heterocycle group is substituted on a ring sulfur, it is substituted with 1 or 2 oxo (═O) group(s); R⁷ is selected from hydrogen, alkyl, perhaloalkyl, and cycloalkyl; R⁸ and R^{8a} are each independently selected from hydrogen, alkyl, and cycloalkyl; and R⁹ is selected from alkyl and cycloalkyl. In some cases, the compound is a compound numbered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240 at pages 17-37 of WO 2018/020474.

In some embodiments of any of the aspects, the MALT-1 inhibitor has a structure of

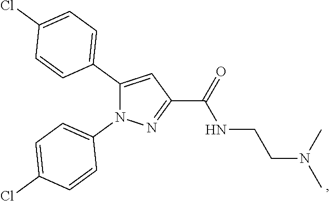 [SMILES: CN(C)CCNC(=O)c1cc(-c2ccc(Cl)cc2)n(-c2ccc(Cl)cc2)n1]

or a pharmaceutically acceptable salt thereof. In some cases, the MALT-1 inhibitor has a structure of

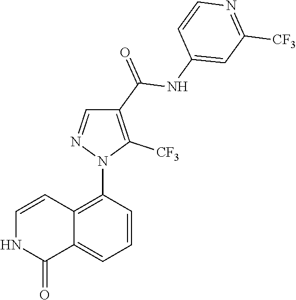 [SMILES: O=C(Nc1ccnc(C(F)(F)F)c1)c1cnn(-c2cccc3c(=O)[nH]ccc23)c1C(F)(F)F]

(JNJ-67856633) or a pharmaceutically acceptable salt thereof.

In some embodiments of any of the aspects, the MALT-1 inhibitor is an inhibitory nucleic acid. Inhibitors of the expression of a given gene (e.g., MALT-1) can be, e.g., an inhibitory nucleic acid. In some embodiments of any of the aspects, the inhibitory nucleic acid is an inhibitory RNA (iRNA), e.g., a siRNA, or a shRNA. Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, e.g., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In some embodiments, an iRNA as described herein effects inhibition of the expression and/or activity of MALT-1. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA (e.g., a CBM signalosome complex or component thereof) level found in the cell without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions.

In some embodiments, the MALT-1 inhibitor is an antisense oligonucleotide. As used herein, an “antisense oligonucleotide” refers to a synthesized nucleic acid sequence that is complementary to a DNA or mRNA sequence, such as that of a microRNA. Antisense oligonucleotides are can be designed to block expression of a DNA or RNA target by binding to the target and halting expression at the level of transcription, translation, or splicing. Antisense oligonucleotides are complementary nucleic acid sequences designed to hybridize under stringent conditions to a gene of interest (e.g., a MALT-1 gene, Genbank Accession No. XM_011525794). For example, an antisense oligonucleotide that inhibits MALT-1 may comprise at least 5, at least 10, at least 15, at least 20, at least 25, at least 30, or more bases complementary to a portion of the coding sequence of the human MALT-1 gene (Genbank Accession No. XM_011525794).

In some embodiments, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

Exemplary embodiments of inhibitory nucleic acids can include, e.g., siRNA, shRNA, miRNA, and/or a miRNA, which are well known in the art, and thus, not described herein.

In some embodiments of any of the aspects, the MALT-1 inhibitor is an siRNA that inhibits activity of MALT-1. One skilled in the art can design siRNA, shRNA, or miRNA to target activity of MALT-1, e.g., using publicly available design tools, such as the siDESIGN Center found on the world wide web at www.dharamacon.gelifesciences.com/design-center/. siRNA, shRNA, or miRNA is commonly made using companies such as Dharmacon (Layfayette, Colo.) or Sigma Aldrich (St. Louis, Mo.). One skilled in the art will be able to readily assess whether the siRNA, shRNA, or miRNA is effective at downregulating the amount of MALT1 protein or the activity of MALT-1, for example by transfecting the siRNA, shRNA, or miRNA into cells and detecting either MALT-1 or its proteolytic targets, such as A20, ReIB, CYLD, BCL10, Regnase, Roquin-1, Roquin-2, HOIL, via Western-blotting (to detect decreased expression levels of MALT1 or decreased levels of its proteolytic targets) or function assays (e.g., measures of T cell function that depend on MALT1, such as IL-2 secretion upon activation with anti-CD3epsilon and anti-CD28 antibodies.

In some embodiments, the MALT-1 inhibitor is an antibody or antigen-binding fragment thereof, or an antibody reagent. As used herein, the term “antibody” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order FR1, CDR1, FR2, CDR2, FR3, CDR3, FR4.

In some embodiments, the antibody binds to an amino acid sequence that corresponds to the amino acid sequence encoding human MALT-1 (Genbank Accession No. XP_011524096). In some embodiments, the anti-MALT-1 antibody binds to an amino acid sequence that comprises the human MALT-1 sequence (Genbank Accession No. XP_011524096) or binds to an amino acid sequence that comprises a sequence with at least 80%, at least 85%, at least 90%, at least 95%, at least 96%, at least 97%, at least 98%, at least 99% or greater sequence identity to the human MALT-1 sequence. In another embodiment, the antibody or antibody reagent binds to an amino acid sequence that comprises a fragment of the human MALT-1 sequence, wherein the fragment is sufficient to bind its target, e.g., MALT-1, and for example, inhibit the function of MALT-1.

In some embodiments, the agent that inhibits activity of MALT-1 is an inhibitory polypeptide. The term “polypeptide” as used herein refers to a polymer of amino acids. The terms “protein” and “polypeptide” are used interchangeably herein. A peptide is a relatively short polypeptide, typically between about 2 and 60 amino acids, 2 and 10 amino acids, 2 and 20 amino acids, 2 and 30 amino acids, 2 and 40 amino acids, 2 and 50 amino acids, 2 and 60 amino acids, 50 and 60 amino acids, 40 and 60 amino acids, 30 and 60 amino acids, 20 and 60 amino acids, 10 and 60 amino acids, 2 and 15, 10 and 30 amino acids, 20 and 50 amino acids, 30 and 60 amino acids, 30 and 40 amino acids, or 40 and 50 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

### Checkpoint Inhibitor

The methods described herein comprise administering a checkpoint inhibitor to the subject. In some embodiments, the checkpoint inhibitor is a small molecule, an inhibitory nucleic acid, an inhibitory polypeptide, antibody or antigen-binding domain thereof, or antibody reagent. In some embodiments, the checkpoint inhibitor is an antibody or antigen-binding domain thereof, or antibody reagent binds an immune checkpoint polypeptide and inhibits its activity. Common checkpoints that are targeted for therapeutics include, but are not limited to PD-L1, PD-L2, PD-1, CTLA-4, TIM-3, LAG-3, VISTA, and TIGIT. In some embodiments, the checkpoint inhibitor is an antibody or antigen-binding domain thereof, or antibody reagent binds a PD-1, PD-L1, or PD-L2 polypeptide and inhibits its activity.

Inhibitors of known checkpoint regulators (e.g., PD-L1, PD-L2, PD-1, CTLA-4, TIM-3, LAG-3, VISTA, or TIGIT) are known in the art. Non-limiting examples of checkpoint inhibitors (with checkpoint targets and manufacturers noted in parentheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MED14736 (PD-L1; AstraZeneca); MSB0010718C (PD-L1; EMD Serono); AUNP12 (PD-1; Aurigene); avelumab (PD-1; Merck); durvalumab (PD-L1; Medimmune); and TSR-022 (TIM3; Tesaro).

In some embodiments, the checkpoint inhibitor inhibits PD-1. PD-1 inhibitors include, but are not limited to Pembrolizumab (Keytruda™), Nivolumab, AUNP-12, and Pidilizumab. In another embodiment, the checkpoint inhibitor inhibits PD-L1. PD-L1 inhibitors include, but are not limited to Atezolizumab, MPDL3280A, Avelumab, and Durvalumab.

Programmed death-ligand 1 (PD-L1; also known as cluster of differentiation 274 (CD274) or B7 homolog 1 (B7-H1)) is a transmembrane protein that functions to suppress the immune system in particular events such as pregnancy, tissue allografts, autoimmune disease, and hepatitis. Binding of PD-L1 to its receptor programmed death-1 (PD-1) transmits an inhibitory signal that reduces the proliferation of T cells and can induce apoptosis. Aberrant PD-L1 and/or PD-1 expression has been shown to promote cancer cell evasion in various tumors. PD-L1/PD-I blockade can be accomplished by a variety of mechanisms including antibodies that bind PD-1 or its ligand, PD-L1. Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, VO2008156712, VO2010089411, VO2010036959, WO2011066342, WO2011159877, VO2011082400, and WO2011161699; which are incorporated by reference herein in their entireties. In certain embodiments, the PD-1 inhibitors include anti-PD-L1 antibodies. PD-1 inhibitors include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade.

### Administration

In some embodiments, the methods described herein relate to treating a subject having or diagnosed as having cancer comprising administering a checkpoint inhibitor and a MALT-1 inhibitor according to the intermittent dosage regimen described herein. As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. melanoma, colon cancer, or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors. A subject can be one who has previously received a treatment or therapy for the condition (e.g., an anti-cancer therapy).

A “subject in need” of treatment for a particular condition can be a subject having that condition, diagnosed as having that condition, or at risk of developing that condition.

In some embodiments, the methods described herein comprise administering an effective amount of a checkpoint inhibitor and a MALT-1 inhibitor according to the intermittent dosage regiment described herein to a subject in order to alleviate a symptom of the cancer. As used herein, “alleviating a symptom of the cancer” is ameliorating any condition or symptom associated with cancer. As compared with an equivalent untreated control, such reduction is by at least 5%, 10%, 20%, 40%, 50%, 60%, 80%, 90%, 95%, 99% or more as measured by any standard technique known to those skilled in the art. The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disease (e.g., melanoma or colon cancer.)

In some embodiments, the agents are administered systemically or locally. In some embodiments, the agents are administered intravenously. In some embodiments, the agents are administered locally, e.g., at the site of the tumor. The route of administration of a MALT-1 inhibitor can be optimized for the type of agent being delivered (e.g., inhibitory nucleic acid, or small molecule), and can be determined by a skilled person. In some embodiments, the MALT-1 inhibitor is administered enterally/gastrointestinally (orally), parenterally, or topically.

The term “effective amount” as used herein refers to the amount of an agent needed to alleviate at least one symptom of the cancer (e.g., headaches). The term “therapeutically effective amount” therefore refers to an amount of an agent that is sufficient to provide a particular anti-cancer effect when administered to a typical subject. An effective amount as used herein, in various contexts, would also include an amount of an agent sufficient to delay the development of a symptom of the cancer, alter the course of a symptom cancer (for example but not limited to, slowing the progression of a cancer), or reverse a symptom of the cancer. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., noninvasive imaging, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, whether to discontinue treatment, resume treatment, or make other alterations to the treatment regimen. The dosage should not be so large as to cause adverse side effects, such as cytotoxic effects. The dosage can also be adjusted by the individual physician in the event of any complication.

The dosage range depends upon the potency, and includes amounts large enough to produce the desired effect, e.g., a decrease in tumor size. Generally, the dosage will vary with the type of agent (e.g., an inhibitory antibody, a small molecule inhibitor of MATL1, or an inhibitory nucleic acid), checkpoint inhibitor, or anti-cancer treatment (e.g., chemotherapeutic), and with the age, sex, and condition of the patient. Typically, the dosage will range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In some embodiments of any of the aspects, the dose range is from 1 μg/kg body weight to 20 μg/kg body weight. Alternatively, the dose range will be titrated to maintain serum levels between 1 μg/mL and 20 μg/mL. In some embodiments, the dosage range is from 1 μg/mL to 15 μg/mL, from 1 μg/mL to 10 μg/mL, from 1 μg/mL to 5 μg/mL, from 1 μg/mL to 2.5 μg/mL, from 2.5 μg/mL to 20 μg/mL, from 5 μg/mL to 20 μg/mL, from 10 μg/mL to 20 μg/mL, from 15 μg/mL to 20 μg/mL, from 10 μg/mL to 5 μg/mL, from 5 μg/mL to 15 μg/mL, from 5 μg/mL to 10 μg/mL, from 2.5 μg/mL to 10 μg/mL, or from 2.5 μg/mL to 15 μg/mL.

Parenteral dosage forms of an agent described herein can be administered to a subject by various routes, including, but not limited to, epidural, intracerebral, intracerebroventricular, epicutaneous, nasal administration, intraarterial, intraarticular, intracardiac, intracavernous injection, intradermal, intralesional, intramuscular, intraocular, intraosseous infusion, intraperitoneal, intrathecal, intrauterine, intravaginal administration, intravenous, intravesical, intravitreal, subcutaneous, transdermal, perivascular administration, or transmucosal. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Intermittent Dosing Regimen

As described in Example 1, combining two dosing regimens for administration of a checkpoint inhibitor and a MALT-1 inhibitor, respectively, leads to an enhanced antitumor effect compared to continuous treatment with the MALT-1 inhibitor.

Described herein is a method of treating cancer in a subject in need thereof comprising administering a checkpoint inhibitor to the subject in combination with intermittent dosing of a MALT-1 inhibitor. The term “intermittent dosing” as used herein refers to non-continuous dosing of a therapeutic agent. For example, a therapeutic agent is administered for a first period of time, followed by a period of time where the therapeutic is withheld (i.e., the therapeutic is not administered) and subsequently followed by a further period of time where the therapeutic is again administered.

In some embodiments, the disclosure provides a method of treating cancer in a subject in need thereof comprising administering a checkpoint inhibitor to the subject; administering a first dose of a MALT-1 inhibitor to the subject for up to seven days; withholding administering the MALT-1 inhibitor to the subject for up to fourteen days after the first dose; and administering a second dose of the MALT-1 inhibitor to the subject after the withholding step. In some embodiments, the checkpoint inhibitor is administered once every three weeks.

In some embodiments, the checkpoint inhibitor and the first dose of the MALT-1 inhibitor administered are administered simultaneously (e.g., on the same day). In some embodiments, the checkpoint inhibitor and the first dose of the MALT-1 inhibitor are administered within about 12 hours to 72 hours of each other (e.g., 12 hours, 16 hours, 24 hours, 36 hours, 48 hours, 60 hours, or 72 hours).

In some embodiments, the first dose of the MALT-1 inhibitor is administered for at least 1 day. In some embodiments, the first dose of the MALT-1 inhibitor is administered for no more than 7 days. In some embodiments, the first dose of the MALT-1 inhibitor is administered for a period of time ranging from 1-7 days (e.g., 1 day, 2 days 3 days, 4 days, 5 days, 6 days or 7 days).

In some embodiments, the method comprises administering a first dose of the MALT-1 inhibitor for 4 days; withholding administering the MALT-1 inhibitor for 3 days; and administering a second dose of the MALT-1 inhibitor. In some embodiments, the method comprises administering a first dose of the MALT-1 inhibitor for 5 days; withholding administering the MALT-1 inhibitor for 5 days; and administering a second dose of the MALT-1 inhibitor. In some embodiments, the method comprises administering a first dose of the MALT-1 inhibitor for 6 days; withholding administering the MALT-1 inhibitor for 6 days; and administering a second dose of the MALT-1 inhibitor. In some embodiments, the method comprises administering a first dose of the MALT-1 inhibitor for 7 days; withholding administering the MALT-1 inhibitor for 7 days; and administering a second dose of the MALT-1 inhibitor. In some embodiments, the method comprises administering a first dose of the MALT-1 inhibitor for 7 days; withholding administering the MALT-1 inhibitor for 14 days; and administering a second dose of the MALT-1 inhibitor.

The methods described herein comprise administering a second dose of the MALT-1 inhibitor. In some embodiments, the second dose of the MALT-1 inhibitor is administered for at least 3 days. In some embodiments, the second dose of the MALT-1 inhibitor is administered for no more than 7 days. In some embodiments, the second dose of the MALT-1 inhibitor is administered for a period of time ranging from 3-7 days (e.g., 3 days, 4 days, 5 days, 6 days or 7 days).

In some embodiments, the methods described herein comprise administering a third dose of the MALT-1 inhibitor. For example, in some embodiments, the third dose of the MALT-1 inhibitor is administered for at least 3 days. In some embodiments, the third dose of the MALT-1 inhibitor is administered for no more than 7 days. In some embodiments, the third dose of the MALT-1 inhibitor is administered for a period of time ranging from 3-7 days (e.g., 3 days, 4 days, 5 days, 6 days or 7 days).

In some embodiments, the method comprises administering a second dose of the MALT-1 inhibitor for 4 days; withholding administering the MALT-1 inhibitor for 3 days; and administering a third dose of the MALT-1 inhibitor. In some embodiments, the method comprises administering a second dose of the MALT-1 inhibitor for 5 days; withholding administering the MALT-1 inhibitor for 5 days; and administering a third dose of the MALT-1 inhibitor. In some embodiments, the method comprises administering a second dose of the MALT-1 inhibitor for 6 days; withholding administering the MALT-1 inhibitor for 6 days; and administering a third dose of the MALT-1 inhibitor. In some embodiments, the method comprises administering a second dose of the MALT-1 inhibitor for 7 days; withholding administering the MALT-1 inhibitor for 7 days; and administering a third dose of the MALT-1 inhibitor.

In various cases, the first dose of the MALT-1 inhibitor and the second dose of the MALT-1 inhibitor are the same dose amount. In other cases, the first dose and the second dose are different dose amounts of the MALT-1 inhibitor. In embodiments where a third dose of the MALT-1 inhibitor is administered, in some cases, the third dose is the same dose amount as the first dose. In some cases, the third dose is the same dose amount as the second dose. In some cases, the third dose is a different dose amount from the first dose.

In some cases, the third dose is a different dose amount from the second dose. In some cases, each of the first dose, the second dose, and the third dose are different dose amounts. In some cases, each of the first dose, the second dose, and the third dose are the same dose amounts.

In any of the embodiments described herein, the checkpoint inhibitor is administered during the duration of the first and second (and subsequent) doses of the MALT-1 inhibitor. In some embodiments, the checkpoint inhibitor is administered once a week (i.e., every 7 days), once every 2 weeks (i.e., every 14 days), once every 3 weeks (i.e., every 21 days), once every 4 weeks (i.e., every 28 days), once every 5 weeks (i.e., every 35 days), once every 6 weeks (i.e., every 42 days) or longer. In some embodiments, the checkpoint inhibitor is administered once every 7, 8, 9, 10 or 11 weeks.

Dosing Holiday

In some cases, the MALT-1 inhibitor is withheld from the subject one more than once occasion during the treatment period with the checkpoint inhibitor (e.g., a three week time period). In particular, after the second dose of the MALT-1 inhibitor for at least three days, administrations of the MALT-1 inhibitor is withheld for at least 4 days. In some cases, administration of the MALT-1 inhibitor is withheld for 5 days. In some cases, it may be that the MALT-1 inhibitor is withheld (i.e., the subject is given a dosing holiday) two, three, four, five, six, seven, or more times in the course of their treatment with the checkpoint inhibitor. The administration of the MALT-1 inhibitor, followed by the dosing holiday, may be referred to as a cycle and in some instances, one, two, three, four, five, six, seven, eight, nine, ten or more such cycles may be used. In other instances, the overall total treatment period may be at least six months, nine months, a year, eighteen months, twenty-four months, or more. It may be that the overall treatment is at least 4, 8, 12, 16, 20, 24, 28, 32, 36, 40, 44, 48 or 52 weeks, or longer, or about such periods. In some instances, where the subject is being treated indefinitely with the checkpoint inhibitor, it may be that the approach of doses with the MALT-1 inhibitor combined with dosing holidays is continued as long as the treatment lasts.

Treatment of Cancer

As used herein, “cancer” refers to a hyperproliferation of cells that have lost normal cellular control, resulting in unregulated growth, lack of differentiation, local tissue invasion, and metastasis. Cancers are classified based on the histological type (e.g., the tissue in which they originate) and their primary site (e.g., the location of the body the cancer first develops), and can be carcinoma, a melanoma, a sarcoma, a myeloma, a leukemia, or a lymphoma. “Cancer” can also refer to a solid tumor. As used herein, the term “tumor” refers to an abnormal growth of cells or tissues, e.g., of malignant type or benign type. “Cancer” can be metastatic, meaning the cancer cells have disseminated from its primary site of origin and migrated to a secondary site.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. melanoma, colon cancer, or other cancer, including cancer resistant to particular therapies, e.g., checkpoint inhibitor therapy. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality.

In some embodiments, the cancer is a carcinoma, a melanoma, a sarcoma, a myeloma, a leukemia, or a lymphoma.

A carcinoma is a cancer that originates in an epithelial tissue. Carcinomas account for approximately 80-90% of all cancers. Carcinomas can affect organs or glands capable of secretion (e.g., breasts, lung, prostate, colon, or bladder). There are two subtypes of carcinomas: adenocarcinoma, which develops in an organ or gland, and squamous cell carcinoma, which originates in the squamous epithelium. Adenocarcinomas generally occur in mucus membranes, and are observed as a thickened plaque-like white mucosa. They often spread easily through the soft tissue where they occur. Squamous cell carcinomas can originate from any region of the body. Examples of carcinomas include, but are not limited to, prostate cancer, colorectal cancer, microsatellite stable colon cancer, microsatellite instable colon cancer, hepatocellular carcinoma, breast cancer, lung cancer, small cell lung cancer, non-small cell lung cancer, lung adenocarcinoma, melanoma, basal cell carcinoma, squamous cell carcinoma, renal cell carcinoma, ductal carcinoma in situ, invasive ductal carcinoma.

Sarcomas are cancers that originate in supportive and connective tissues, for example bones, tendons, cartilage, muscle, and fat. Sarcoma tumors usually resemble the tissue in which they grow. Non-limiting examples of sarcomas include, Osteosarcoma or osteogenic sarcoma (originating from bone), Chondrosarcoma (originating from cartilage), Leiomyosarcoma (originating from smooth muscle), Rhabdomyosarcoma (originating from skeletal muscle), Mesothelial sarcoma or mesothelioma (originate from membranous lining of body cavities), Fibrosarcoma (originating from fibrous tissue), Angiosarcoma or hemangioendothelioma (originating from blood vessels), Liposarcoma (originating from adipose tissue), Glioma or astrocytoma (originating from neurogenic connective tissue found in the brain), Myxosarcoma (originating from primitive embryonic connective tissue), or Mesenchymous or mixed mesodermal tumor (originating from mixed connective tissue types).

Melanoma is a type of cancer forming from pigment-containing melanocytes. Melanoma typically develops in the skin, but can occur in the mouth, intestine, or eye.

Myelomas are cancers that originate in plasma cells of bone marrow. Non-limiting examples of myelomas include multiple myeloma, plasmacytoma and amyloidosis.

Leukemias (also known as “blood cancers”) are cancers of the bone marrow, which is the site of blood cell production. Leukemia is often associated with the overproduction of immature white blood cells. Immature white blood cells do not function properly, rendering the patient prone to infection. Leukemia additionally affects red blood cells, and can cause poor blood clotting and fatigue due to anemia. Leukemia can be classified as being acute myeloid leukemia (AML), Chronic myeloid leukemia (CML), Acute lymphocytic leukemia (ALL), and Chronic lymphocytic leukemia (CLL). Examples of leukemia include, but are not limited to, Myelogenous or granulocytic leukemia (malignancy of the myeloid and granulocytic white blood cell series), Lymphatic, lymphocytic, or lymphoblastic leukemia (malignancy of the lymphoid and lymphocytic blood cell series), and Polycythemia vera or erythremia (malignancy of various blood cell products, but with red cells predominating).

Lymphomas develop in the glands or nodes of the lymphatic system (e.g., the spleen, tonsils, and thymus), which purifies bodily fluids and produces white blood cells, or lymphocytes. Unlike leukemia, lymphomas form solid tumors. Lymphoma can also occur in specific organs, for example the stomach, breast, or brain; this is referred to as extranodal lymphomas). Lymphomas are subclassified into two categories: Hodgkin lymphoma and Non-Hodgkin lymphoma. The presence of Reed-Stemberg cells in Hodgkin lymphoma diagnostically distinguishes Hodgkin lymphoma from Non-Hodgkin lymphoma. Non-limiting examples of lymphoma include Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL). In one embodiment, the cancer is DLBCL or Follicular lymphoma.

In some embodiments, the cancer is a solid tumor. Non-limiting examples of solid tumors include Adrenocortical Tumor, Alveolar Soft Part Sarcoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carcinomas.

In various embodiments, the cancer is metastatic.

In various cases, the subject being treated using a method as disclosed herein suffers from a solid tumor or a soluble cancer with a microtumor environment. In various cases, the cancer is melanoma, head and neck cancer, lung cancer (e.g., non-small cell lung cancer), bladder cancer, kidney cancer, prostate cancer, a central nervous system (CNS) cancer, breast cancer, stomach cancer, thyroid cancer, ovarian cancer, or Non-Hodgkin's lymphoma. In some cases, the cancer is melanoma. In various cases, the cancer is bladder cancer. In various cases, the cancer is kidney cancer. In various cases, the cancer is non-small cell lung cancer. In various cases, the cancer is head and neck cancer.

### Further Optional Combination Therapy

In various embodiments, the combination therapy disclosed herein with the specific dosing schedules can be combined with therapy of a third therapeutic agent, e.g., an additional anti-cancer therapy. An anti-cancer therapy can be, e.g., chemotherapy, radiation therapy, chemo-radiation therapy, immunotherapy, hormone therapy, surgery or stem cell therapy.

In accordance with some embodiments, the subject is administered a chemotherapeutic agent in combination with the methods described herein. Exemplary chemotherapeutic agents include, but are not limited to, a platinum chemotherapeutic agent, an anthracyclin therapeutic agent, or an alkylating chemotherapeutic agent. Non-limiting examples of chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCy®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere), doxorubicin hydrochloride (Adriamycin®, Rubex®), etoposide (Vepesid®), fludarabine phosphate (Fludara®), 5-fluorouracil (AdruciM®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (IR,2R,45)-4-[(2R)-2 [(1R,95,125,15R,16E,18R,19R,21R,235,24E,26E,28Z,305,325,35R)-I,18-dihydroxy-19,30-dimethoxy-15,17,21,23,29,35-hexamethyl-2,3,10,14,20-pentaoxo-I I,36-dioxa-4-azatricyclo[30.3.1.04′9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOI); rapamycin (AY22989, Sirolimus®); simapimod (CAS 164301-51-3); emsirolimus, (5-{2,4-Bis[(35,)-3-methylmorpholin-4-yl]pyrido[2,3-(i]pyrmidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[I,4-dioxo-4-[[4-(4-oxo-8-phenyl-4H-I-benzopyran-2-yl)morpholinium-4-yl]methoxy]butyl]-L-arginylglycyl-L-a-aspartylL-serne-, inner salt (SF1126, CAS 936487-67-1), and XL765. Exemplary immunomodulators include, e.g., afutuzumab (available from Roche®)); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velbani); and vinorelbine (Navelbine®). Exemplary proteosome inhibitors include bortezomib (Velcade®); carfilzomib (PX-171-007, (5)-4-Methyl-N-((5)-I-(((5)-4-methyl-I-((R)-2-methyloxiran-2-yl)-I-oxopentan-2-yl)amino)-I-oxo-3-phenylpropan-2-yl)-2-((5,)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(IIS′)-2-[(2R)-2-methyl-2-oxiranyl]-2-oxo-I-(phenylmethyl)ethyl]-L-serinamide (ONX-0912).

One of skill in the art can readily identify a chemotherapeutic agent of use with methods and compositions describe herein (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In accordance with some embodiments, the subject is administered a radiation therapy in combination with the methods described herein. Radiation therapy, according to the invention disclosed herein, encompasses both non-invasive (external) and invasive (internal) radiation therapies. In an external radiation therapy, treatment is affected by radiation sources outside the body, whereas in an invasive radiation therapy treatment is affected by radiation sources planted inside the body. The representative diseases treated by non-invasive or invasive radiation therapy include, for example, cancer, rheumatoid arthritis, angioplasty, or restenosis.

In accordance with some embodiments, the subject is administered a chemo-radiation therapy, e.g., a combination of a chemotherapy and radiation therapy, in combination with the methods described herein.

In accordance with some embodiments, the subject is administered an immunotherapy in combination with the methods described herein. As used herein, “immunotherapy” refers to a treatment designed, e.g., to enhance the function of the immune system of a subject or to use transfer of immune cells or of immune molecules (e.g., cytokines) to stop or slow the growth of cancer cells, stop the metastasis of cancer cells, and/or target the cancer cells for cell death in the subject. Exemplary immunotherapies include a monoclonal antibody, a non-specific immunotherapy, an oncolytic virus therapy, adoptive T-cell therapy (e.g., adoptive CD4⁺ or CD8⁺ effector T cell therapy), adoptive natural killer (NK) cell therapy, adoptive NK T cell therapy, CAR T cell therapy and cancer (e.g., tumor) vaccines.

In accordance with some embodiments, the subject is administered a non-specific immunotherapy in combination with the methods described herein. Two common non-specific immunotherapies include, e.g., interferons and interleukins. Interferons (such as Roferon-A [2α], Intron A [2β], Alferon [2α]) boost the immune system to target cancer cells for programmed cell death, and/or slow the growth of cancer cells. Interleukins (such as interleukin-2, IL-2, or aldesleukin (Proleukin)) boost the immune system to produce cells that target cancer cells for programmed cell death. Interleukins are used to treat, e.g., kidney cancer and skin cancer, including melanoma.

In accordance with some embodiments, the subject is administered an oncolytic virus in combination with the methods described herein. Oncolytic virus therapy utilizes a genetically modified virus (e.g., a herpes simplex virus, or other virus) to target cancer cells for programmed cell death via an immune response. An oncolytic virus is administered locally, e.g., injected into a tumor, where the virus enters the cancer cells and replicates. The replication can result in lysis of the cancer cells, resulting in the release of antigens and activating an immune response that targets the cancer cells for programmed cell death. Administration of the virus can be repeated until the desired effect is obtained (e.g., the tumor is eradicated). Oncolytic virus therapy (e.g., talimogene laherparepvec (Imlygic), or T-VEC) has been approved for treatment of melanoma.

In some embodiments, a subject having cancer is administered an engineered T cell in combination with the methods described herein. T cell therapy utilizes T cell that have been engineered express an exogenous chimeric antigen receptor (CAR). As used herein, “chimeric antigen receptor” or “CAR” refers to an artificially constructed hybrid polypeptide comprising an antigen-binding domain (e.g., an antigen-binding portion of an antibody (e.g., a scFV)), a transmembrane domain, and a T-cell signaling and/or T-cell activation domain (e.g., intracellular signaling domain). CARs have the ability to redirect T-cell specificity and reactivity toward a selected target in a non-MHC-restricted manner, exploiting the antigen-binding properties of monoclonal antibodies. Further discussion of CARs can be found, e.g., in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

In some embodiments, a subject having cancer is administered a CAR T cell that targets a tumor antigen on the cell surface of a tumor cell in combination with the methods described herein. As used herein, the terms “tumor antigen” refers to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively.

In some embodiments, a subject having cancer is administered an CAR T cell that targets EGFR (Epidermal growth factor receptor) on non-small cell lung cancer, epithelial carcinoma, glioma; EGFRvIII (Variant III of the epidermal growth factor receptor) on glioblastoma; HER2 (Human epidermal growth factor receptor 2) on ovarian cancer, breast cancer, glioblastoma, colon cancer, osteosarcoma, medulloblastoma; MSLN (Mesothelin) on mesothelioma, ovarian cancer, pancreatic adenocarcinoma; PSMA (Prostate-specific membrane antigen) on prostate cancer; CEA (Carcinoembryonic antigen) on pancreatic adenocarcinoma, breast cancer, colorectal carcinoma; GD2 (Disialoganglioside 2) on neuroblastoma, melanoma; IL13Rα2 (Interleukin-13Rα2) on glioma; GPC3 (Glypican-3) on hepatocellular carcinoma; CAIX (Carbonic anhydrase IX) on renal cell carcinoma (RCC); L1-CAM (L1 cell adhesion molecule) on neuroblastoma, melanoma, ovarian adenocarcinoma; CA125 (Cancer antigen 125, also known as MUC16) on epithelial ovarian cancers; CD133 (Cluster of differentiation 133, also known as prominin-1) on glioblastoma, cholangiocarcinoma (CCA); FAP (Fibroblast activation protein) on malignant pleural mesothelioma (MPM); CTAG1B (Cancer/testis antigen 1B, also known as NY-ESO-1) on melanoma and ovarian cancer; MUC1 (Mucin 1) on seminal vesicle cancer; FR-α (Folate receptor-α) on ovarian cancer in combination with the methods described herein.

In some embodiments, a subject having cancer is administered a CAR T cell that targets a checkpoint inhibitor in combination with the methods described herein. In one embodiment, a subject having cancer is administered an anti-PD-1 CAR T cell. In one embodiment, a subject having cancer is administered an anti-PD-L1 CAR T cell in combination with the methods described herein.

In some embodiments, a subject having cancer is administered a cancer vaccine in combination with the methods described herein. Cancers that can be treated with and/or prevented by cancer vaccines include but are not limited to bladder cancer, brain tumors, breast cancer, cervical cancer, colorectal cancer, kidney cancer, leukemia, lung cancer, melanoma, myeloma, pancreatic cancer, and prostate cancer.

In some embodiments, the subject having cancer is administered an adoptive T cell therapy in combination with the methods described herein. Exemplary T cells that can be used in adoptive T cell therapy include CD4⁺ or CD8⁺ effector T cell, regulatory T cells, or cytolytic T cells.

In some embodiments, the subject having cancer is administered an adoptive NK cell therapy in combination with the methods described herein. Natural killer (NK) cells are immune cells that function to target a cancer cell for programmed cell death without requiring prior sensitization to a tumor antigen. NK target cancer cells through a variety of mechanisms, e.g., through receptor-mediated cytotoxicity. NK cells express a germ-line encoded receptors, such as the c-type lectin homodimer, NKG2D, which binds to stress induced ligands (e.g., ULBP's, MICA/MICB) expressed on tumor cells. Upon ligation, NK cells degranulate, releasing perforin and granzymes to induce target cell apoptosis. NK cell degranulation can also be triggered though a process called antibody dependent cell-mediated cytotoxicity (ADCC). NK cells and T cells can be modified (e.g., with cytokines such as IL-2, IL-12, IL-15, or IL-18) to increase their cancer cell capabilities and specificity. NK cells administered to a subject can be autologous or allogeneic. NK cells administered to a subject can be expand in vivo or ex vivo. Cancers that can be treated with adoptive NK cell or T cell therapy include, but are not limited to advanced melanoma, renal cell carcinoma, acute myeloid leukemia, lymphoma, solid tumors, non-Hodgkin's lymphoma, chronic lymphocytic leukemia, non-B lineage hematologic malignancies, Her2⁺ breast cancer, and Her2⁺ gastric cancer. The use of adoptive NK cell and adoptive NK T cell therapies are further reviewed in, e.g., Davis, Z B, et al. Cancer J. 2015 November-December; 21(6): 486-491, which is incorporated by reference herein in its entirety.

In some embodiments, adoptive T cell therapy, e.g., CD4⁺ or CD8⁺ effector T cell therapy, or NK T cell therapy, is reactive with tumor antigens. T cells for adoptive T cell therapies can be are purified from, e.g., tumor tissue, blood, or other patient tissue. Purified T cells can be e.g., activated, expanded, and/or genetically modified, e.g., ex vivo in cell culture. Activated, expanded, and/or genetically modified T cells can be e.g., administered into the patient, for example, by intravenous injection or other acceptable routes, in combination with methods described herein.

In accordance with some embodiments, the subject is administered a hormone therapy in combination with the methods described herein. Hormone therapy is designed to add, block, or remove hormones from the body to, e.g., halt or slow the growth of cancer cells. Hormone therapy can include administration of, e.g., progesterone, oophorectomy, tamoxifen, gonadotropin-releasing hormone (GnRH) agonists or analogues and androgen therapy. Hormone therapy can also refer to removing glands, e.g., thyroid, pancreas, and ovary, to reduce the levels of hormones in the body. Hormone therapies are known in the art and can be administered by a skilled person.

In accordance with some embodiments, the subject is administered a stem cell therapy in combination with the methods described herein. Stem cell therapy can comprise removing a subjects stem cells prior to receiving treatment to destroy all stem cells (e.g., chemotherapy, radiotherapy, or a combination thereof). Stems cells can be re-administered to the patient following such treatment (e.g., a stem cell transplant). A stem cell transplant can be autologous, or allogenic. A stem cell transplant can be a tandem transplant (e.g., two or more transplants in a row), a mini-transplant (e.g., a subject's immune system is suppressed less than a typical transplant), or a syngeneic stem cell transplant (e.g., allogenic stem cells received from an identical twin). Cancers that can be treated with stem cell therapy include but are not limited to leukemias, lymphomas, multiple myeloma, testicular cancer, neuroblastoma, and certain childhood cancers.

### Efficacy

The efficacy of the treatment methods described herein can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom (e.g., headaches, or bone pain), and/or the incidence of a condition treated according to the methods described herein or any other measurable parameter appropriate. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein.

The technology described herein is further illustrated by the following examples which in no way should be construed as being further limiting.

Some embodiments of the technology described herein can be defined according to any of the following numbered paragraphs:

1. A method of treating cancer in a subject in need thereof comprising:
(a) administering a checkpoint inhibitor to the subject;
(b) administering a first dose of a MALT-1 inhibitor to the subject for up to 7 days;
(c) withholding administering the MALT-1 inhibitor for at least 2 days after the administration of (b); and
(d) administering a second dose of the MALT-1 inhibitor to the subject after the withholding step.

2. The method of paragraph 1, wherein the checkpoint inhibitor is an anti-TIM3 antibody, an anti-TIGIT antibody, an anti-VISTA antibody, an anti-LAG3 antibody, an anti-NKG2A antibody, an anti-PD1 antibody, an anti-PD-L1 antibody or an anti-PD-L2 antibody.

3. The method of paragraph 2, wherein the checkpoint inhibitor is an anti-PD1 antibody.

4. The method of paragraph 3, wherein the anti-PD1 antibody is Pembrolizumab (Keytruda), Nivolumab, AUNP-12 or Pidilizumab.

5. The method of paragraph 2, wherein the checkpoint inhibitor is an anti-PDL1 antibody.

6. The method of paragraph 5, wherein the anti-PDL1 antibody is Atezolizumab, MPDL3280A, Avelumab or Durvalumab.

7. The method of paragraph 3, wherein the anti-PD1 antibody is administered once every three weeks.

8. The method of any one of paragraphs 1-7, wherein the withholding step comprises withholding the MALT-1 inhibitor for up to 14 days.

9. The method of any one of paragraphs 1-7, wherein (b) comprises administering the MALT-1 inhibitor for 5 consecutive days.

10. The method of any one of paragraphs 1-8, wherein (c) comprises withholding the MALT-1 inhibitor for 5 consecutive days.

11. The method of any one of paragraphs 1-10, wherein the MALT-1 inhibitor is a small molecule.

12. The method of paragraph 11, wherein the MALT-1 inhibitor is MI-2 or an analog thereof, MI-2A1, MI-2A2, MI-2A3, MI-2A4, MI-2A5, MI-2A6, MI-2A7, a pyrazolo pyrimidine derivative, a phenothiazine derivative, a thiazolo-pyridine derivative, or tetrapeptide Z-VRPR-FMK, or a pharmaceutically acceptable salt thereof.

13. The method of paragraph 12, wherein the MALT-1 inhibitor is mepazine, thioridazine, or promazine, or a pharmaceutically acceptable salt thereof.

14. The method of any one of paragraphs 1-11, wherein the MALT-1 inhibitor is

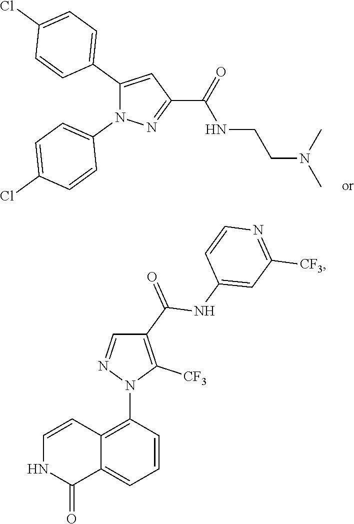 [SMILES: CN(C)CCNC(=O)c1cc(-c2ccc(Cl)cc2)n(-c2ccc(Cl)cc2)n1 ; O=C(Nc1ccnc(C(F)(F)F)c1)c1cnn(-c2cccc3c(=O)[nH]ccc23)c1C(F)(F)F]

or a pharmaceutically acceptable salt thereof.

15. The method of any one of paragraphs 1-11, wherein the MALT-1 inhibitor is (S)-mepazine or a pharmaceutically acceptable salt thereof.

16. The method of any one of paragraphs 1-15, wherein the cancer is a carcinoma, a melanoma, a sarcoma, a myeloma, a leukemia, or a lymphoma.

17. The method of paragraph 16, wherein the cancer is a melanoma or colon cancer.

18. The method of any of paragraphs 1-17, wherein cancer is a solid tumor.

19. The method of paragraph 18, wherein the solid tumor is an Adrenocortical Tumor, an Alveolar Soft Part Sarcoma, a Chondrosarcoma, a Colorectal Carcinoma, a Desmoid Tumors, a Desmoplastic Small Round Cell Tumor, an Endocrine Tumors, an Endodermal Sinus Tumor, an Epithelioid Hemangioendothelioma, a Ewing Sarcoma, a Germ Cell Tumors (Solid Tumor), a Giant Cell Tumor of Bone and Soft Tissue, a Hepatoblastoma, a Hepatocellular Carcinoma, a Melanoma, a Nephroma, a Neuroblastoma, a Non-Rhabdomyosarcoma Soft Tissue Sarcoma (NRSTS), an Osteosarcoma, a Paraspinal Sarcoma, a Renal Cell Carcinoma, a Retinoblastoma, a Rhabdomyosarcoma, a Synovial Sarcoma, or a Wilms Tumor.

### EXAMPLES

### Example 1—Interval Versus Continuous Mepazine Treatment in Combination with Anti-PD-1 Checkpoint Inhibition Therapy for Melanoma

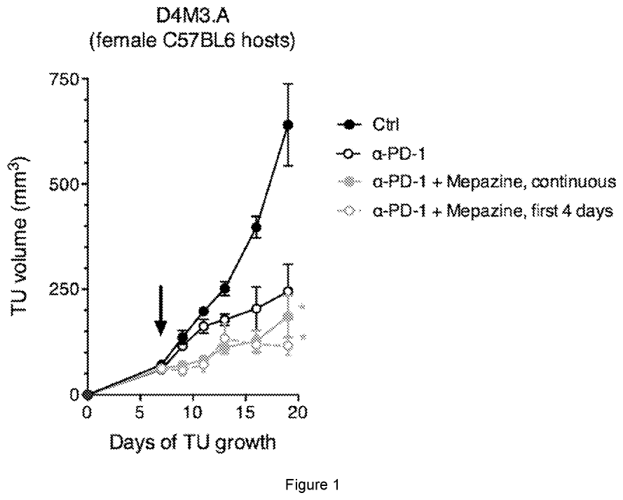

Female C57BL/6 mice were implanted subcutaneously with 10⁶ D4M.3A melanoma cells. Starting 7 days later (arrow), animals were treated with daily i.p. injections of 200 μg of anti-PD-1 mAbs (clone 29F.1A12) or isotype control mAb (rat IgG2a). Some animals in addition received daily i.p. injections of 16 mg/kg bodyweight of the allosteric MALT-1 inhibitor mepazine, either for the first 4 days (days 7-10) only, or continuously until the end of the experiment on day 19. Tumor size was measured using a caliper every 2-3 days. n=4/group. *=p<0.05 versus Ctrl. As shown in FIG. 1, interval treatment with mepazine is at least as effective as continuous treatment.

### Example 2—Efficacy Study

Female C57BL/6 mice are implanted subcutaneously with 10⁶ MC38 colon cancer cells. The mice are put into 29 separate Groups (n=10 per group) as follows:

Group 1 to 9: (S)-Mepazine (9 dose levels, 0.125-32 mg/kg BW)+aPD-1

Group 10 to 18: Mepazine racemate (9 dose levels, 0.125-32 mg/kg BVV)+aPD-1

Group 19 to 27: (S)-Mepazine monotherapy (9 dose levels, 0.125-32 mg/kg BVV)

Group 28: αPD-1 monotherapy

Group 29: No treatment

Starting 12 days later, animals are administered 3-5 doses (tbc) of 200 μg aPD-1 i.p. BIW; 2 doses of (S)-Mepazine or Mepazine racemate i.p. or p.o. daily. The dosing continues for three weeks (three weeks of dosing and one week of observation post last dose). Body weight and tumor volume measurement will be assessed on days 12, 14, 16, 19 and every 3 days from thereon.

### Example 3—Once Daily Versus Twice Daily Dosing of Mepazine in Combination with Anti-PD-1 Checkpoint Inhibition Therapy for Colon Cancer

Female C57BL/6 mice are implanted subcutaneously with 10⁶ MC38 colon cancer cells. The mice are put into 6 separate Groups (n=10 per group) as follows:

Group 1: (S)-Mepazine OD low dose

Group 2: (S)-Mepazine OD high dose

Group 3: (S)-Mepazine BID low dose

Group 4: (S)-Mepazine BID high dose

Group 5: αPD-1 monotherapy

Group 6: No treatment

Starting 12 days later, animals are administered 3-5 doses (tbc) of 200 μg αPD-1 i.p.; and doses of either 1, 2, 4, 6, 8, 10, 12, 16, 32 mg/kg bodyweight (S)-Mepazine or Mepazine racemate i.p. or p.o. on days on days 12, 14, 16, 18, 20. The dosing continues for three weeks (three weeks of dosing and one week of observation post last dose). Body weight and tumor volume measurement will be assessed twice weekly.

### Example 4-Dosing Regimen of (S)-Mezapine in Combination with Anti-PD-1 Checkpoint Inhibitory Therapy for Colon Cancer

Female C57BL/6 mice are implanted subcutaneously with 10⁶ MC28 cancer cells. The mice are put into 8 separate Groups (n=10 per group) as follows:

Group 1: (S)-Mepazine dosed every day of 3-week cycle at optimal dose; αPD-1 continuous q.a.d.

Group 2: (S)-Mepazine dosing per 3-week cycle consists of daily dosing (days 1-7) and no dosing for 2 weeks (days 8-21) at optimal dose; αPD-1 continuous q.a.d.

Group 3: (S)-Mepazine dosing per 3-week cycle consists of daily dosing for first 3 days of a week (days 1-3, 8-10, 15-17) and no dosing for last 4 days of the week (days 4-7, 11-14, 18-21) at optimal dose; αPD-1 continuous q.a.d.

Group 4: (S)-Mepazine dosed every day of 3-week cycle at supra-optimal dose; αPD-1 continuous q.a.d.

Group 5: (S)-Mepazine dosing per 3-week cycle consists of daily dosing (days 1-7) and no dosing for 2 weeks (days 8-21) at supra optimal dose; αPD-1 continuous q.a.d.

Group 6: (S)-Mepazine dosing per 3-week cycle consists of daily dosing for first 3 days of a week (days 1-3, 8-10, 15-17) and no dosing for last 4 days of the week (days 4-7, 11-14, 18-21) at supra optimal dose; continuous αPD-1 q.a.d.

Group 7: Continuous αPD-1 treatment.

Group 8: No treatment.

Starting 12 days later, animals are administered doses of (s)-mepazine (of either 1, 2, 4, 6, 8, 10, 12, 16, 32 mg/kg bodyweight) and/or αPD-1 as noted above. The dosing continues for three weeks (three weeks of dosing and one week of observation post last dose). Body weight and tumor volume measurement will be assessed twice weekly.

### Example 5—Efficacy Study

Female C57BL/6 mice are implanted subcutaneously with 10⁶ B16F10 melanoma cells. The mice are put into 4 separate Groups (n=10 per group) as follows:

Group 1: (S)-Mepazine+αPD-1

Group 2: (S)-Mepazine

Group 3: αPD-1

Group 4: No treatment

Starting 10 days later, mice will receive continuous dosing of 200 μg αPD-1 i.p. q.a.d. together with doses of either 1, 2, 4, 6, 8, 10, 12, 16, 32 mg/kg bodyweight (S)-mepazine. The dosing continues for three weeks (three weeks of dosing and one week of observation post last dose). Body weight and tumor volume measurement will be assessed on days 12, 14, 16, 19 and every 3 days thereon.

### Example 6—Efficacy Study

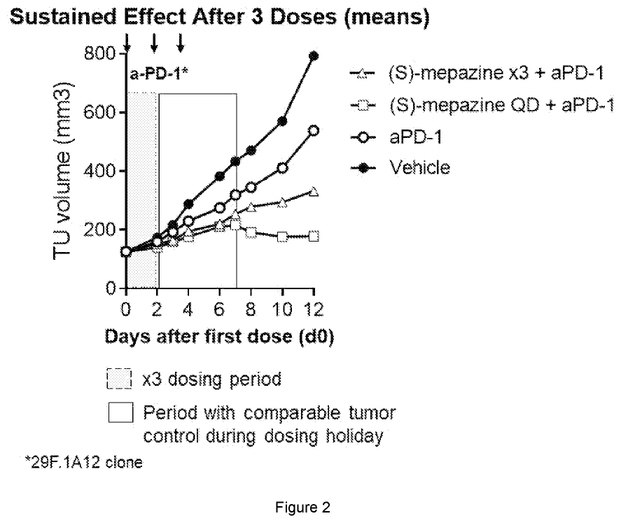

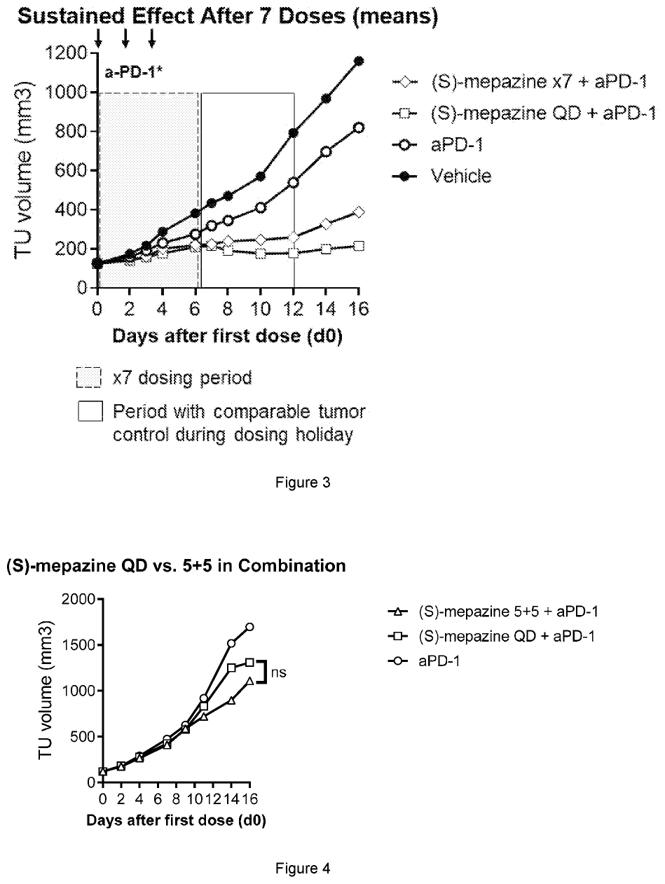

Male C57BL/6 mice were implanted subcutaneously with D4M.3A syngeneic tumors. The mice were dosed with (S)-mepazine IP and with anti-PD1 mouse clone 29F.1A12 dosed 3 times 200 μg/dose at days 0, 2, and 4 of a treatment cycle. A ×3 arm of mice were dosed at days 0, 1, and 3 of a 21 day treatment cycle with (S)-mepazine IP. A ×7 arm of mice were dosed at days 0-6 of a 21 day treatment cycle with (S)-mepazine IP. A QD arm of mice were dosed daily with (S)-mepazine IP. Tumor volume was measured each day. The results of the study are shown in FIGS. 2 and 3, for ×3 arm and ×7 arm, respectively. These results show that both the ×3 and ×7 arms showed improved activity against the tumor compared to anti-PD1 alone, and provided efficacy for tumor control up to 7 days after the (S)-mepazine was dosed, comparable to the QD dosing arm.

### Example 7—Efficacy Study

Male C57BL/6 mice were implanted subcutaneously with D4M.3A syngeneic tumors. The mice were dosed with (S)-mepazine PO and with anti-PD1 mouse clone 29F.1A12 dosed 3 times 200 μg/dose at days 0, 2, and 4 of a treatment cycle. The mice were dosed 5 days on with (S)-mepazine and 5 days off (a dose holiday) (5+5) or each day with (S)-mepazine (QD) PO. Tumor volume was measured each day. The results of the study are shown in FIG. 4, showing that the 5+5 dosing method was equivalent to a QD dosing schedule.

What is claimed is:

1. A method of treating cancer in a subject in need thereof comprising:
(a) administering a checkpoint inhibitor to the subject;
(b) administering a first dose of a MALT-1 inhibitor to the subject for up to 7 days;
(c) withholding administering the MALT-1 inhibitor for at least 2 days after the administration of (b); and
(d) administering a second dose of the MALT-1 inhibitor to the subject after the withholding step.

2. The method of claim 1, wherein the checkpoint inhibitor is an anti-TIM3 antibody, an anti-TIGIT antibody, an anti-VISTA antibody, an anti-LAG3 antibody, an anti-NKG2A antibody, an anti-PD1 antibody, an anti-PD-L1 antibody or an anti-PD-L2 antibody.

3. The method of claim 2, wherein the checkpoint inhibitor is an anti-PD1 antibody.

4. The method of claim 3, wherein the anti-PD1 antibody is Pembrolizumab (Keytruda), Nivolumab, AUNP-12 or Pidilizumab.

5. The method of claim 2, wherein the checkpoint inhibitor is an anti-PDL1 antibody.

6. The method of claim 5, wherein the anti-PDL1 antibody is Atezolizumab, MPDL3280A, Avelumab or Durvalumab.

7. The method of claim 3, wherein the anti-PD1 antibody is administered once every three weeks.

8. The method of any one of claims 1-7, wherein the withholding step comprises withholding the MALT-1 inhibitor for up to 14 days.

9. The method of any one of claims 1-7, wherein (b) comprises administering the MALT-1 inhibitor for 5 consecutive days.

10. The method of any one of claims 1-8, wherein (c) comprises withholding the MALT-1 inhibitor for 5 consecutive days.

11. The method of any one of claims 1-10, wherein the MALT-1 inhibitor is a small molecule.

12. The method of claim 11, wherein the MALT-1 inhibitor is MI-2 or an analog thereof, MI-2A1, MI-2A2, MI-2A3, MI-2A4, MI-2A5, MI-2A6, MI-2A7, a pyrazolo pyrimidine derivative, a phenothiazine derivative, a thiazolo-pyridine derivative, or tetrapeptide Z-VRPR-FMK, or a pharmaceutically acceptable salt thereof.

13. The method of claim 12, wherein the MALT-1 inhibitor is mepazine, thioridazine, or promazine, or a pharmaceutically acceptable salt thereof.

14. The method of any one of claims 1-11, wherein the MALT-1 inhibitor is 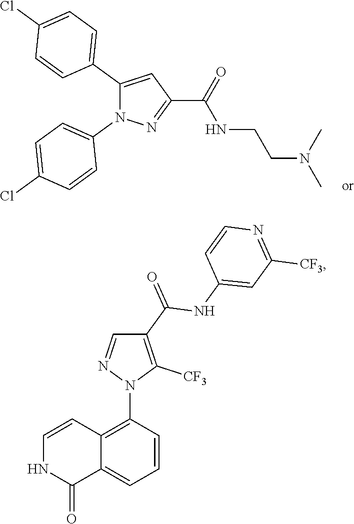 [SMILES: CN(C)CCNC(=O)c1cc(-c2ccc(Cl)cc2)n(-c2ccc(Cl)cc2)n1 ; O=C(Nc1ccnc(C(F)(F)F)c1)c1cnn(-c2cccc3c(=O)[nH]ccc23)c1C(F)(F)F]
or a pharmaceutically acceptable salt thereof.

15. The method of any one of claims 1-11, wherein the MALT-1 inhibitor is (S)-mepazine or a pharmaceutically acceptable salt thereof.

16. The method of any one of claims 1-15, wherein the cancer is a carcinoma, a melanoma, a sarcoma, a myeloma, a leukemia, or a lymphoma.

17. The method of claim 16, wherein the cancer is a melanoma or colon cancer.

18. The method of any of claims 1-17, wherein cancer is a solid tumor.

19. The method of claim 18, wherein the solid tumor is an Adrenocortical Tumor, an Alveolar Soft Part Sarcoma, a Chondrosarcoma, a Colorectal Carcinoma, a Desmoid Tumors, a Desmoplastic Small Round Cell Tumor, an Endocrine Tumors, an Endodermal Sinus Tumor, an Epithelioid Hemangioendothelioma, a Ewing Sarcoma, a Germ Cell Tumors (Solid Tumor), a Giant Cell Tumor of Bone and Soft Tissue, a Hepatoblastoma, a Hepatocellular Carcinoma, a Melanoma, a Nephroma, a Neuroblastoma, a Non-Rhabdomyosarcoma Soft Tissue Sarcoma (NRSTS), an Osteosarcoma, a Paraspinal Sarcoma, a Renal Cell Carcinoma, a Retinoblastoma, a Rhabdomyosarcoma, a Synovial Sarcoma, or a Wilms Tumor.
